(19) United States
(12) Patent Application Publication
(10) Pub. No.: US 2022/0282319 A1
(43) Pub. Date: Sep. 8, 2022

(54) ANALYTE DETECTION IN SITU USING NUCLEIC ACID ORIGAMI

(71) Applicant: 10x Genomics, Inc., Pleasanton, CA (US)

(72) Inventor: Toon VERHEYEN, Solna (SE)

(21) Appl. No.: 17/685,016

(22) Filed: Mar. 2, 2022

Related U.S. Application Data
(60) Provisional application No. 63/156,276, filed on Mar. 3, 2021.

(51) Int. Cl. C12Q 1/6841

(52) U.S. Cl. CPC C12Q 1/6841

(57) ABSTRACT

The present disclosure relates in some aspects to methods and compositions, and kits for in situ analysis of nucleic acid targets in a biological sample using nucleic acid origami. In some aspects, the nucleic acid origami and methods disclosed herein allow detection of a target nucleic acid in a sample without requiring amplification.

### CROSS-REFERENCE TO RELATED APPLICATIONS

This application claims priority to U.S. Provisional Patent Application No. 63/156,276, filed Mar. 3, 2021, entitled “ANALYTE DETECTION IN SITU USING NUCLEIC ACID ORIGAMI,” which is herein incorporated by reference in its entirety for all purposes.

### FIELD

The present disclosure relates in some aspects to methods and compositions for in situ analysis of an analyte in a biological sample using nucleic acid origami.

### BACKGROUND

Single molecule fluorescent in situ hybridization (smFISH), including amplified smFISH methods such as hybridization chain reaction (HCR), are used to determine expression levels of analytes, such as RNA. A major limitation of these approaches is that the signals may be dim while background fluorescence may be concomitantly high (especially in samples such as FFPE samples) with large variability depending on the tissue type, sample age, and fixation conditions. Imaging at high magnification (e.g., 60×-100×) is usually required. As a result, only a very small area of the sample is usually imaged (typically, 40-50 fields of view), thus limiting the ability to detect target analyte variability across a sample. Thus, improved methods are needed. The present disclosure addresses this and other needs.

### SUMMARY

In some aspects, provided herein is a method for analyzing a biological sample, comprising: a) contacting a biological sample comprising a plurality of cells with a nucleic acid scaffold and a binding staple, wherein a nucleic acid origami comprising the nucleic acid scaffold and the binding staple is formed, and wherein the binding staple comprises a binding region that directly or indirectly binds to a nucleic acid molecule in the biological sample; and b) detecting the nucleic acid origami in the biological sample, thereby analyzing localization of the nucleic acid molecule in the biological sample.

In some embodiments, the nucleic acid origami can be pre-formed prior to the contacting step, and the contacting step can comprise contacting the biological sample with the pre-formed nucleic acid origami.

In any of the preceding embodiments, the nucleic acid origami can comprise a folded core comprising the nucleic acid scaffold, and the binding region can protrude from the folded core.

In any of the preceding embodiments, the nucleic acid origami can be contacted with a detection staple directly or indirectly labelled with a detectable moiety. In some instances, the detectable moiety is a fluorophore. In some instances, the detection staple is covalently or non-covalently coupled to the detectable moiety. In some instances, the 3′ end and/or the 5′ end of the detection staple is coupled to the detectable moiety. In some embodiments, the nucleic acid origami can comprise the detection staple. In some embodiments, the nucleic acid origami comprising the detection staple is pre-formed prior to the contacting step. In some embodiments, the nucleic acid scaffold forms a folded core of the nucleic acid origami and the detection staple comprises a detection region protruding from the folded core. In some embodiments, the detection region comprises a 5′ end of the detection staple. In some embodiments, the detection region comprises a 3′ end of the detection staple.

In any of the preceding embodiments, the detection region may directly hybridize to a detectably labelled oligonucleotide.

In any of the preceding embodiments, the detection region may directly hybridize to an adapter which directly or indirectly binds to a detectably labelled oligonucleotide. In some instances, the adapter comprises a toehold region that does not hybridize to the detection region or the detectably labelled oligonucleotide.

In any of the preceding embodiments, the detectably labelled oligonucleotide can be directly or indirectly bound to the nucleic acid origami prior to the contacting step.

In any of the preceding embodiments, the method can further comprise contacting the biological sample with the detectably labelled oligonucleotide and/or the adapter prior to, during, or after contacting the sample with the nucleic acid origami.

In any of the preceding embodiments, the detection staple can be covalently coupled to the detectable moiety. In some instances, the detection staple does not comprise a region that does not hybridize to the nucleic acid origami.

In any of the preceding embodiments, the detecting step can comprise detecting a signal from the detectable moiety and/or the detectably labelled oligonucleotide, thereby detecting the nucleic acid origami.

In any of the preceding embodiments, the nucleic acid origami can comprise a plurality of detection staples. In some instances, the nucleic acid origami comprises at least 5, at least 10, at least 15, at least 20, at least 25, at least 30, at least 35, at least 40, at least 45, or at least 50 detection staples.

In any of the preceding embodiments, the binding region can directly hybridize to the nucleic acid molecule in the biological sample.

In any of the preceding embodiments, the binding region can indirectly bind to the nucleic acid molecule in the biological sample. In some embodiments, the binding region directly hybridizes to an adapter which directly or indirectly binds to the nucleic acid molecule in the biological sample. In some instances, the nucleic acid molecule in the biological sample is a rolling circle amplification product. In some embodiments, the adapter comprises a sequence complementary to the binding region and a sequence complementary to the nucleic acid molecule in the biological sample.

In any of the preceding embodiments, the binding region can comprise a 5′ end and/or a 3′ end of the binding staple.

In any of the preceding embodiments, the nucleic acid origami can comprise a plurality of binding staples. In some instances, the binding region of a first binding staple comprises a 5′ end of the first binding staple, and the binding region of a second binding staple comprises a 3′ end of the second binding staple.

In some embodiments, the binding region of the first binding staple can comprise a 5′ end phosphate group. In some embodiments, the binding region of the second binding staple can comprise a ribonucleotide. In some instances, the ribonucleotide is the 3′ terminal nucleotide of the second binding staple. In some embodiments, the binding regions of the first and second binding staples can hybridize to adjacent sequences of the nucleic acid molecule in the biological sample to form a hybridization complex, wherein the 5′ end of the first binding staple and the 3′ end of the second binding staple are brought in proximity to each other by the nucleic acid molecule. In some embodiments, the method can further comprise ligating the 5′ end of the first binding staple and the 3′ end of the second binding staple, with or without gap filling prior to the ligation.

In any of the preceding embodiments, the binding staple can comprise a staple region that hybridizes to the nucleic acid scaffold. In some instances, the binding staple can comprise a linker linking the staple sequence and the binding region. In some instances, the binding region is between about 10 and about 50 nucleotides in length. In some instances, the binding region is between about 15 and about 25 nucleotides in length. In some instances, the binding region is about 20 nucleotides in length. In some instances, the linker is between about 1 and about 10 nucleotides in length. In some instances, the linker is between about 2 and about 5 nucleotides in length.

In any of the preceding embodiments, an anchor and the binding region of the binding staple can be hybridized to adjacent sequences in the nucleic acid molecule. In some instances, the anchor and the binding region are ligated using the nucleic acid molecule or a splint as template, with or without gap filling prior to the ligation. In some embodiments, the hybridization and/or the ligation can be performed in situ.

In any of the preceding embodiments, the nucleic acid molecule can be an endogenous DNA or RNA molecule in the biological sample.

In any of the preceding embodiments, the nucleic acid molecule in the biological sample can be a viral or cellular DNA or RNA or a product thereof. In some embodiments, the product is a hybridization product, a ligation product, an extension product (e.g., by a DNA or RNA polymerase), a replication product, a transcription/reverse transcription product, and/or an amplification product such as a rolling circle amplification (RCA) product.

In any of the preceding embodiments, the nucleic acid molecule in the biological sample can be comprised in a labelling agent that directly or indirectly binds to an analyte in the biological sample, or can be comprised in a product of the labelling agent. In some embodiments, the product is a hybridization product, a ligation product, an extension product (e.g., by a DNA or RNA polymerase), a replication product, a transcription/reverse transcription product, and/or an amplification product such as a rolling circle amplification (RCA) product. In some embodiments, the labelling agent can comprise a reporter oligonucleotide. In some instances, the reporter oligonucleotide comprises one or more barcode sequences and the product of the labelling agent comprises one or a plurality of copies of the one or more barcode sequences.

In any of the preceding embodiments, the nucleic acid molecule in the biological sample can be a rolling circle amplification (RCA) product of a circular or circularizable (e.g., padlock) probe or probe set that hybridizes to a DNA (e.g., a cDNA of an mRNA) or RNA molecule in the biological sample.

In any of the preceding embodiments, the RCA products of a plurality of different mRNA and/or cDNA molecules can be analyzed, a barcode sequence in a particular circular or circularizable (e.g., padlock) probe or probe set can uniquely correspond to a particular mRNA or cDNA molecule, and the particular circular or circularizable (e.g., padlock) probe or probe set can further comprise an anchor sequence that is common among circular or circularizable (e.g., padlock) probes or probe sets for a subset of the plurality of different mRNA and/or cDNA molecules.

In any of the preceding embodiments, the labelling agent can comprise a binding moiety that directly or indirectly binds to a non-nucleic acid analyte in the biological sample, and the reporter oligonucleotide in the labelling agent identifies the binding moiety and/or the non-nucleic acid analyte. In some embodiments, the non-nucleic acid analyte comprises a peptide, a protein, a carbohydrate, and/or lipid, In some embodiments, the binding moiety of the labelling agent can comprise an antibody or antigen binding fragment thereof that directly or indirectly binds to a protein analyte, and the nucleic acid molecule in the biological sample can be a rolling circle amplification (RCA) product of a circular or circularizable (e.g., padlock) probe or probe set that hybridizes to a reporter oligonucleotide of the labelling agent.

In any of the preceding embodiments, the nucleic acid molecule can be an RCA product generated in situ. In any of the preceding embodiments, the nucleic acid molecule can be immobilized in the biological sample. In any of the preceding embodiments, the nucleic acid molecule can be crosslinked to one or more other molecules (e.g., a cellular molecule or an extracellular molecule) in the biological sample, a matrix such as a hydrogel, and/or one or more functional groups on a substrate.

In some aspects, provided herein is a method for analyzing a biological sample, comprising: a) contacting a biological sample comprising a plurality of cells with a nucleic acid origami, wherein: the nucleic acid origami comprises a nucleic acid scaffold, a binding staple, and a plurality of fluorescently labelled detection staples, and the binding staple comprises a binding region that directly or indirectly binds to a nucleic acid molecule in the biological sample; and b) detecting fluorescent signals from the plurality of fluorescently labelled detection staples of the nucleic acid origami in the biological sample, thereby analyzing localization of the nucleic acid molecule in the biological sample. In some embodiments, one or more of the detection staples can be covalently coupled to a fluorophore.

In some embodiments, one or more of the detection staples can comprise a detection region protruding from a folded core comprising the nucleic acid scaffold of the nucleic acid origami. In some embodiments, the detection region can directly hybridize to a fluorescently labelled oligonucleotide, or the detection region can directly hybridize to an adapter which directly or indirectly binds to a fluorescently labelled oligonucleotide.

In any of the preceding embodiments, the nucleic acid molecule can be a rolling circle amplification (RCA) product.

In some embodiments, the binding region can directly hybridize to the RCA product, or the binding region can directly hybridize to an adapter which directly or indirectly binds to the RCA product.

In some embodiments, the RCA product can comprise a barcode sequence corresponding to an analyte in the biological sample. In some embodiments, the method can further comprise analyzing the barcode sequence using sequential hybridization, sequencing by hybridization, sequencing by ligation, sequencing by synthesis, sequencing by binding, or any combination thereof.

In any of the preceding embodiments, the RCA product can be immobilized in the biological sample. In some instances, the RCA product is crosslinked to one or more other molecules (e.g., a cellular molecule or an extracellular molecule) in the biological sample, a matrix such as a hydrogel, and/or one or more functional groups on a substrate.

In some aspects, provided herein is a method for analyzing a biological sample, comprising: a) contacting a biological sample comprising a plurality of cells with a plurality of nucleic acid origami, wherein: the biological sample comprises a plurality of nucleic acid molecules, each nucleic acid origami comprises a nucleic acid scaffold, a binding staple, and a plurality of fluorescently labelled detection staples, wherein the binding staple comprises (i) a staple region that hybridizes to the nucleic acid scaffold, and (ii) a binding region that hybridizes to an adapter which in turn hybridizes to a target sequence in the plurality of nucleic acid molecules; and b) detecting fluorescent signals from the fluorescently labelled detection staples, thereby analyzing localization of the plurality of nucleic acid molecules in the biological sample. In some instances, the binding staple comprises (iii) a linker linking the staple region and the binding region. In some embodiments, the plurality of nucleic acid molecules can comprise rolling circle amplification (RCA) products. In some embodiments, each RCA product can comprise multiple copies of a barcode sequence corresponding to an analyte of interest. In some embodiments, the adapter can comprise a sequence that hybridizes to the barcode sequence, whereby the adapter corresponds to the analyte of interest.

In any of the preceding embodiments, the detection staples of a first nucleic acid origami and a second nucleic acid origami can be labelled with a first fluorophore and a second fluorophore, respectively.

In any of the preceding embodiments, the binding staples of the first nucleic acid origami and the second nucleic acid origami share the same staple region. In any of the preceding embodiments, the binding staples of the first nucleic acid origami and the second nucleic acid origami can comprise different binding regions.

In any of the preceding embodiments, the first and second fluorophores can be different, wherein the binding regions of the first and second nucleic acid origami are different and each corresponds to the first or second fluorophore, respectively.

In any of the preceding embodiments, the method can further comprise repeating step (a) and step (b) sequentially one or more times. In some embodiments, the method comprises sequential detecting of two or more nucleic acid origami at a position in the repeated steps (b) and the detected signals are used to build a signal code sequence corresponding to localization of the nucleic acid molecule at the position in the biological sample. In some embodiments, the signal code sequence comprises a plurality of detected fluorescent signals from the plurality of fluorescently labelled detection staples of the nucleic acid origami. In any of the preceding embodiments, the signal code sequence may correspond to a barcode sequence for the nucleic acid molecule. In any of the preceding embodiments, each nucleic acid origami can comprise the same nucleic acid scaffold. In any of the preceding embodiments, each nucleic acid origami can comprise the same staple region. In any of the preceding embodiments, two or more of the plurality of nucleic acid origami can comprise different binding regions.

In some aspects, provided herein is a method for analyzing a biological sample, comprising: a) contacting a biological sample with n sets of probes for n target sequences T1, . . . , Tk, . . . , Tn, in m cycles, wherein: Probe Set 1 comprises P11, . . . , P1j, . . . , and P1m; Probe Set k comprises Pk1, . . . , Pkj, . . . , and Pkm; Probe Set n comprises Pn1, . . . , Pnj, . . , and Pnm; j, k, m, and n are integers, 2≤j≤m, and 2≤k≤n; the biological sample is contacted with Probe Library P11, . . . , Pk1, . . . , and Pn1 in Cycle 1, with Probe Library P1j, . . . , Pkj, . . . , and Pnj in Cycle j, and Probe Library P1m, Pkm, . . . , and Pnm in Cycle m, and each probe comprises (i) a target hybridization sequence that hybridizes to T1, . . . , Tk, . . . , Tn, respectively, and (ii) an adapter sequence for binding to a nucleic acid origami, wherein the nucleic acid origami comprises a binding staple comprising a staple region that hybridizes to the nucleic acid scaffold and a binding region that directly or indirectly hybridizes to the adapter sequence, and a plurality of fluorescently labelled detection staples; b) in a particular cycle, contacting the biological sample with a plurality of nucleic acid origami that hybridize to the probes contacted with the biological sample in the particular cycle, wherein fluorescent signals from the nucleic acid origami for probes in different probe sets or different probe libraries are of the same or different colors; and c) detecting fluorescent signals from the nucleic acid origami in the biological sample, thereby generating a signal code sequence over the m cycles for each target sequence and analyzing localization of molecules comprising the n target sequences in the biological sample.

In some embodiments, the molecules comprising the n target sequences can be rolling circle amplification (RCA) products.

In any of the preceding embodiments, T1, . . . , Tk, . . . , and Tn can comprise barcode sequences B1, Bk, . . . , and Bn, respectively, each corresponding to an analyte of interest. In some embodiments, the analytes of interest can comprise DNA, RNA, and/or protein molecules.

In any of the preceding embodiments, the plurality of nucleic acid origami can comprise the same nucleic acid scaffold.

In any of the preceding embodiments, the plurality of nucleic acid origami can comprise the same staple region.

In any of the preceding embodiments, the plurality of nucleic acid origami can comprise different binding regions. In some embodiments in a particular nucleic acid origami, the binding region can correspond to the fluorescent labels in the detection staples. In some instances, the plurality of nucleic acid origami comprise 3, 4, 5, or more different fluorescent labels.

In any of the preceding embodiments, the detecting step can comprise: i) contacting the biological sample with a plurality of hybridization chain reaction (HCR) or linear oligo hybridization chain reaction (LO-HCR) monomers, wherein: one or more HCR or LO-HCR monomers are detectably labelled, the detection region comprises or is directly or indirectly coupled to an initiator sequence that hybridizes to an HCR or LO-HCR monomer of the plurality to initiate an HCR or LO-HCR, and an HCR or LO-HCR complex comprising the one or more detectably labelled HCR or LO-HCR monomers is generated; and ii) detecting a signal from the HCR or LO-HCR complex in the biological sample.

In some embodiments, the HCR or LO-HCR can be a linear or non-linear HCR, e.g., a branched HCR or LO-HCR. In some embodiments, the HCR or LO-HCR can be in one dimension or in multiple dimensions.

In any of the preceding embodiments, the plurality of HCR or LO-HCR monomers can comprise one or more linear nucleic acid molecules and/or one or more hairpin nucleic acid molecules.

In any of the preceding embodiments, the one or more detectably labelled HCR or LO-HCR monomers can be covalently or noncovalently coupled to a fluorophore.

In any of the preceding embodiments, the one or more detectably labelled HCR or LO-HCR monomers can be covalently or noncovalently coupled to a nucleic acid origami. In some instances, the nucleic acid origami is covalently or noncovalently coupled to a fluorophore.

In any of the preceding embodiments, one detectably labelled HCR or LO-HCR monomer can serve as a splint that hybridizes to two or more detectably labelled HCR or LO-HCR monomers. In some embodiments, the splint can comprise a toehold region that does not hybridize to the two or more detectably labelled HCR or LO-HCR monomers.

In some embodiments, one or more of the plurality of HCR or LO-HCR monomers may not be detectably labelled. In some embodiments, one HCR or LO-HCR monomer that is not detectably labelled can serve as a splint that hybridizes to two or more detectably labelled HCR or LO-HCR monomers. In some embodiments, the splint can comprise a toehold region that does not hybridize to the two or more detectably labelled HCR or LO-HCR monomers.

In any of the preceding embodiments, the detection region can comprise the initiator sequence.

In any of the preceding embodiments, the detection region can hybridize to an adapter which hybridizes to an initiator comprising the initiator sequence. In some embodiments, the adapter can comprise a toehold region that does not hybridize to the detection region or the initiator. In any of the preceding embodiments, the toehold region can be between about 5 and about 20 nucleotides in length, e.g., about 10 nucleotides in length.

In any of the preceding embodiments, the adapter and/or the initiator hybridized thereto can be dissociated from the detection strand in the absence of a denaturing agent. In some instances, the denaturing agent is formamide. In some embodiments, the dissociation can comprise contacting the biological sample with a nucleic acid that hybridizes to the toehold region and displaces the adapter from the detection region.

In any of the preceding embodiments, the detection region can hybridize to an adapter which comprises the initiator sequence. In some embodiments, the method can comprise hybridizing a first adapter comprising a first initiator sequence to the detection region, wherein the first adapter hybridizes to a first sequence in the detection region. In some embodiments, a first HCR or LO-HCR complex comprising the first adapter can be generated.

In any of the preceding embodiments, the method can further comprise hybridizing a second adapter comprising a second initiator sequence to the detection region, wherein the second adapter hybridizes to a second sequence in the detection region.

In some embodiments, the first and second sequences in the detection region can be overlapping sequences, and hybridization of the second adapter can displace the first adapter from the detection region. In some embodiments, a second HCR or LO-HCR complex comprising the second adapter can be generated.

In any of the preceding embodiments, the biological sample can be a processed or cleared biological sample. In any of the preceding embodiments, the biological sample can be a tissue sample. In any of the preceding embodiments, the tissue sample can be a tissue slice between about 1 μm and about 50 μm in thickness. In some instances, the tissue slice is between about 5 μm and about 35 μm in thickness. In any of the preceding embodiments, the tissue sample can be embedded in a hydrogel.

In one aspect, disclosed herein is a kit for analyzing a biological sample, comprising a plurality of nucleic acid origami, wherein each nucleic acid origami comprises a nucleic acid scaffold, a binding staple, and a plurality of modified staples, wherein the binding staple comprises (i) a staple region that hybridizes to the nucleic acid scaffold, and (ii) a binding region configured to hybridize to an adapter which is configured to hybridize to a target sequence in a nucleic acid molecule present or suspected of being present in the biological sample. In some instances, the binding staple comprises (iii) a linker linking the staple region and the binding region. In some embodiments, a modified staple comprises a detectable label and/or a protruding region that does not bind to the nucleic acid scaffold. In some embodiments, the protruding region is between about 10 and about 60 nucleotides in length. In some embodiments, the protruding region is between about 15 and about 35 nucleotides in length. In some embodiments, the protruding region is about 20 nucleotides in length.

In one aspect, disclosed herein is a kit for analyzing a biological sample, comprising a nucleic acid scaffold, a binding staple, and a detection staple, wherein the binding staple comprises a binding region that directly or indirectly binds to a nucleic acid molecule in the biological sample and a staple region that binds to the nucleic acid scaffold, wherein detection staple can be directly or indirectly labelled with a detectable moiety, wherein the nucleic acid scaffold is capable of forming a nucleic acid origami with the binding staple and the detection staple. In some instances, the kit comprises instructions for analyzing the biological sample according to a method disclosed herein. In some embodiments, the detection staple is covalently or non-covalently coupled to the detectable moiety. In some instances, the 3′ end and/or the 5′ end of the detection staple is coupled to the detectable moiety. In some embodiments, the detection staple comprises a detection region and a staple region, the staple region binds to the nucleic acid scaffold, and the nucleic acid scaffold forms a folded core and the detection region protrudes from the folded core.

In any of the preceding embodiments, the kit can further comprise instructions for forming the nucleic acid origami, wherein the nucleic acid origami comprises a folded core comprising the nucleic acid scaffold, and the binding region protrudes from the folded core.

In any of the preceding embodiments, the detection region can comprise a 5′ end or 3′ end of the detection staple. In some instances, the kit further comprises (i) a detectably labelled oligonucleotide capable of hybridizing to the detection region or (ii) an adapter capable of hybridizing to the detection region and a detectably labelled oligonucleotide that directly or indirectly binds to the adapter. In some instances, the adapter comprises a toehold region that does not hybridize to the detection region or the detectably labelled oligonucleotide. In any of the preceding embodiments, the kit can comprise a plurality of binding staples. In some instances, the binding region of a first binding staple comprises a 5′ end of the first binding staple, and the binding region of a second binding staple comprises a 3′ end of the second binding staple. In any of the preceding embodiments, the binding staple can comprise a staple region that hybridizes to the nucleic acid scaffold. In some instances, the binding staple can comprise a linker linking the staple sequence and the binding region. In some instances, the binding region is between about 10 and about 50 nucleotides in length. In some instances, the binding region is between about 15 and about 25, optionally about 20, nucleotides in length. In some instances, the linker is between about 1 and about 10 nucleotides in length. In some instances, the linker is between about 2 and about 5 nucleotides in length.

### BRIEF DESCRIPTION OF THE DRAWINGS

FIG. 1A shows an exemplary nucleic acid origami binding to a target sequence via one or more binding staples (one binding staple is shown). The nucleic acid origami can be a spherical DNA origami that functions as a probe for binding a nucleic acid such as a rolling circle product (RCP) and may comprise a plurality of detection staples for enhanced signals, wherein each detection staple is directly or indirectly labelled with a detectable moiety. In some instances, the detectable moiety is a fluorophore.

FIG. 1B shows an exemplary nucleic acid origami binding to a target sequence via one or more binding staples (two binding staples are shown). The nucleic acid origami can be a spherical DNA origami that functions as a probe for binding a nucleic acid such as an RNA, DNA, cDNA, or RCP, and may comprise a plurality of protruding detection staples as binding sites for other probes or probe sets.

FIG. 2A shows an exemplary nucleic acid origami comprising a detection staple comprising a detection region hybridized to a detectably labelled probe. The detection staple may have a protruding 5′ end (shown) or a protruding 3′ end (not shown).

FIG. 2B shows an exemplary nucleic acid origami comprising a detection staple comprising a detection region hybridized to an adapter, which is in turn hybridized to a detectably labelled probe. The detection staple may have a protruding 5′ end (shown) or a protruding 3′ end (not shown).

FIGS. 2C-2D each shows an exemplary nucleic acid origami comprising a detection staple that serves as an initiator for assembling a hybridization complex, such as a linear oligo-hybridization chain reaction (LO-HCR) complex. The detection staple may have a protruding 5′ end (shown) or a protruding 3′ end (not shown).

FIG. 3 shows an exemplary nucleic acid origami comprising protruding staples (indicated by thick black lines) positioned in pairs in Recognition site 1 or Recognition site 2, wherein each of the two sites comprises one protruding staple protruding at its 5′ end and a second protruding staple protruding from its 3′ end.

FIG. 4A shows a first exemplary nucleic acid origami comprising a first binding staple comprising a protruding 3′ end that, via a first adapter, binds to Recognition site 1 on a nucleic acid (e.g., an RCP). FIG. 4B shows a second exemplary nucleic acid origami comprising a second binding staple comprising a protruding 5′ end that, via a second adapter, binds to Recognition site 2 which overlaps with Recognition site 1 on the nucleic acid. The overlapping recognition sites allow the first nucleic acid origami/first adapter complex and the second nucleic acid origami/second adapter complex to displace one another from the same target sequence (e.g., a barcode sequence in an RCP) in different hybridization cycles. The same nucleic acid origami can be used in different cycles, e.g., the first and second binding staples can be part of the same nucleic acid origami.

FIG. 5 shows an exemplary nucleic acid origami, wherein a 5′ protruding staple has a phosphate group at its 5′ end, and a 3′ protruding staple has an RNA base at its 3′ end. The two protruding staples hybridize to the target sequence in such a fashion as to position the 5′ phosphate group next to the 3′ RNA base for ligation.

FIG. 6A shows an exemplary nucleic acid origami hybridizing directly to a target sequence (e.g., an RCP) via a binding staple.

FIG. 6B shows an exemplary nucleic acid origami hybridizing to a target sequence (e.g., an RCP) via an adapter probe that hybridizes to the binding staple.

FIG. 7 shows an exemplary signal enhancement workflow described herein. During the sequencing cycles, the adapter probes are hybridized. Then, nucleic acid origami probes are added to hybridize to the adapter probes on the RCPs.

FIG. 8A shows an exemplary LO-HCR reaction using a nucleic acid origami as a scaffold for analyte detection (e.g., in situ sequencing), wherein the protruding end of a detection staple serves as an initiator that hybridizes to a LO-HCR monomer and initiates the LO-HCR reaction. LO-HCR complexes can be assembled on the protruding ends of each of the detection staples.

FIG. 8B shows an exemplary HCR reaction using a nucleic acid origami as a scaffold for analyte detection (e.g., in situ sequencing), wherein the protruding end of a detection staple serves as an initiator that hybridizes to an HCR monomer (a metastable hairpin) and initiates the HCR reaction. HCR complexes can be assembled on the protruding ends of each of the detection staples.

FIG. 9 shows an example of a multiplex assay (e.g., greater than 4-5 different analytes), wherein the protruding detection region of a detection staple hybridizes to an adapter, and the detection region and/or the adapter may comprise analyte-specific barcode sequence(s).

FIG. 10 shows an example of a multiplex assay (e.g., greater than 4-5 different analytes), wherein the protruding detection region of a detection staple comprises overlapping recognition sites for adapters. A first adapter hybridizes to a first recognition site on the detection staple and initiates the assembly of a hybridization complex (e.g., via HCR or LO-HCR; LO-HCR is shown) in one cycle. A signal from the hybridization complex is detected, and a second adapter hybridizes to a second recognition site which partially overlaps with first recognition site and displaces the hybridized first adapter and hybridization complex bound thereto. The second adapter initiates a new cycle of hybridization complex assembling process (e.g., via HCR or LO-HCR; LO-HCR is shown). The detection region of the nucleic acid origami and/or the first and/or second adapters may comprise analyte-specific barcode sequence(s).

FIG. 11A shows gel electrophoresis results of four exemplary origami-padlocks, each corresponding to a unique recognition site of the PCP4 mRNA molecule.

FIG. 11B shows microscope images showing the detection of an exemplary Cy7-labelled origami-padlock in a tissue sample.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods involving the use of nucleic acid origami-based probes for analyzing one or more target nucleic acid(s), such as a target nucleic acid (for example, a messenger RNA or an RCP product) present in a cell or a biological sample. Also provided are polynucleotides, sets of polynucleotides, compositions, and kits for use in accordance with the provided methods.

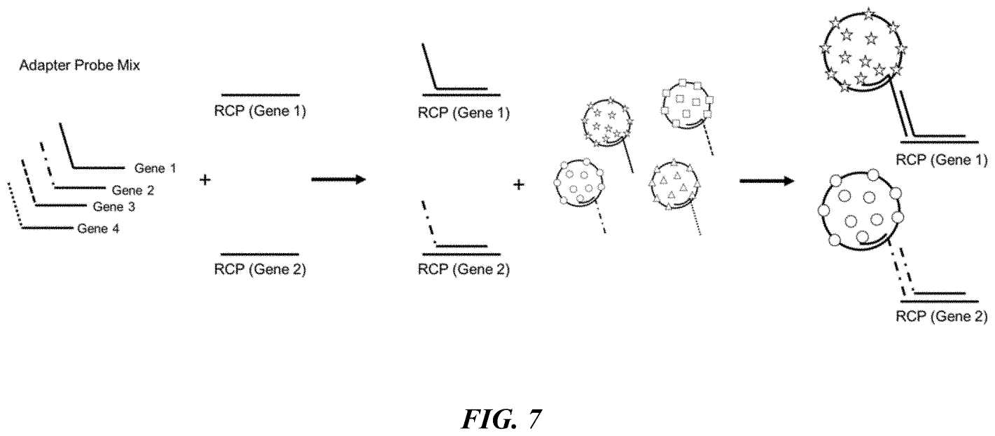

In some aspects, the provided methods and nucleic acid origami probes can provide a method of signal enhancement (e.g., the signal is enhanced by a theoretical n-fold compared to a conventional detectably labelled probe, wherein n is the number of detectably labelled detection staples). For example, the nucleic acid origami probe can occupy the binding site of an original fluorescent oligo in a conventional RCP detection assay (e.g., via direct or indirect binding), effectively replacing a single detectable moiety with n detectable oligos (e.g., as shown in FIG. 7). In this way, the structure can be used as a signal amplifier. This can be particularly useful as it potentially means that RCPs can be detected on a 10× or even 5× objective, which would speed up imaging time dramatically and allow imaging of a larger sample area.

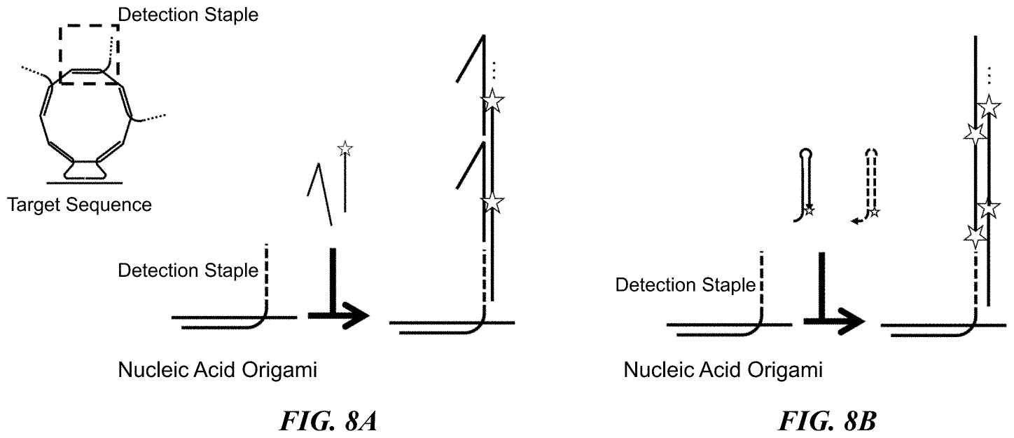

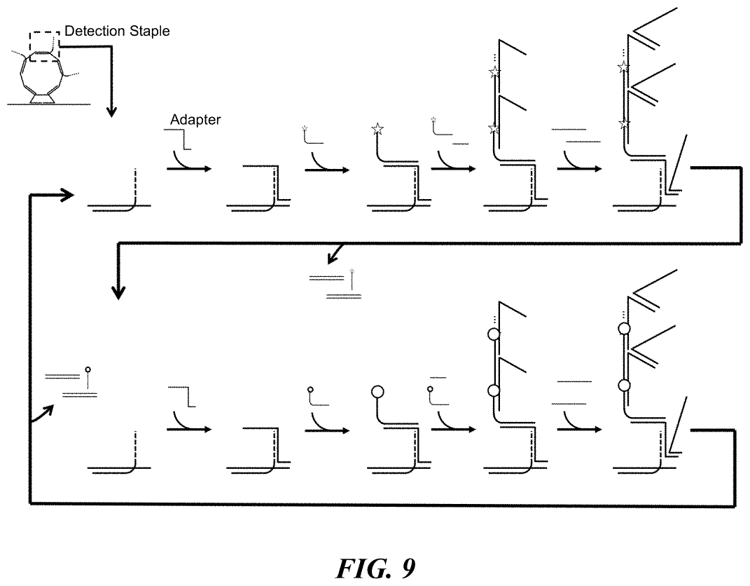

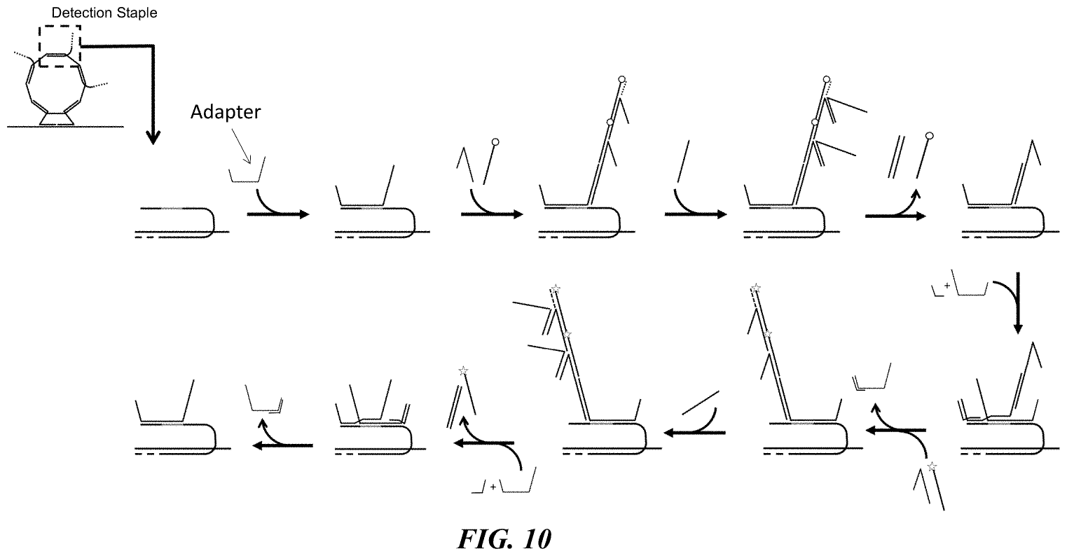

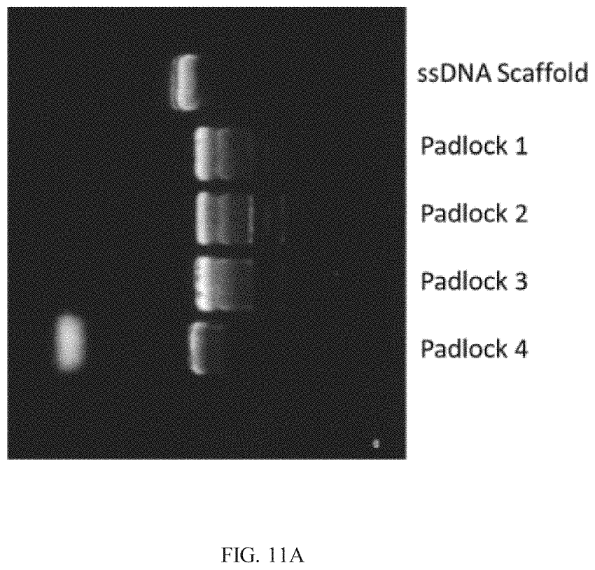

In some aspects, the provided methods and nucleic acid origami probes can be used as an alternative to existing methods that require amplification, such as the generation of RCPs via rolling circle amplification. In some embodiments, the nucleic acid origami comprises detection staples having protruding detection regions, wherein the detection region can comprise or be linked to a barcode sequence for sequential decoding hybridizations (e.g., as shown in FIGS. 9 and 10). In some embodiments, the detection region can comprise or be linked to an initiator for assembly of a hybridization complex for signal amplification such as via a hybridization chain reaction (HCR) (e.g., as shown in FIG. 8B) or a linear-oligo hybridization chain reaction (LO-HCR) (e.g., as shown in FIG. 8A). Thus, a plurality of detection staples in a nucleic acid origami probe can provide sufficient signal enhancement for direct detection of a nucleic acid analyte (e.g., an mRNA molecule) without the need for an enzymatic (e.g., catalyzed by a polymerase) amplification, such as a rolling circle amplification.

Furthermore, in some embodiments, binding staples can be designed in pairs, wherein one binding staple has a protruding 3′ end, and the other binding staple has a protruding 5′ end. In some embodiments, the two protruding ends of a pair of binding staples can hybridize to a target nucleic acid such that they can be ligated by template ligation, providing additional positional stability to the nucleic acid origami probe. In some embodiments, the binding region of the first binding staple can comprise a 5′ end phosphate group. In some embodiments, the binding region of the second binding staple can comprise a ribonucleotide, e.g., as the 3′ terminal nucleotide. In some embodiments, the binding regions of the first and second binding staples can hybridize to adjacent sequences of the nucleic acid molecule in the biological sample to form a hybridization complex, wherein the 5′ end of the first binding staple and the 3′ end of the second binding staple are brought in proximity to each other by the nucleic acid molecule. In some embodiments, the method can further comprise ligating the 5′ end of the first binding staple and the 3′ end of the second binding staple, with or without gap filling prior to the ligation.

In some embodiments, provided herein is a method for analyzing a biological sample, comprising: a) contacting a biological sample comprising a plurality of cells with a nucleic acid scaffold and a binding staple, wherein a nucleic acid origami comprising the nucleic acid scaffold and the binding staple is formed, and wherein the binding staple comprises a binding region that directly or indirectly binds to a nucleic acid molecule in the biological sample; and b) detecting the nucleic acid origami in the biological sample, thereby analyzing localization of the nucleic acid molecule in the biological sample.

In some embodiments, provided herein is a method for analyzing a biological sample, comprising: a) contacting a biological sample comprising a plurality of cells with a plurality of nucleic acid origami, wherein: the biological sample comprises a plurality of nucleic acid molecules, each nucleic acid origami comprises a nucleic acid scaffold, a binding staple, and a plurality of fluorescently labelled detection staples, wherein the binding staple comprises (i) a staple region that hybridizes to the nucleic acid scaffold, (ii) a binding region that hybridizes to an adapter which in turn hybridizes to a target sequence in the plurality of nucleic acid molecules, and optionally (iii) a linker linking the staple region and the binding region; and b) detecting fluorescent signals from the fluorescently labelled detection staples, thereby analyzing localization of the plurality of nucleic acid molecules in the biological sample.

In some embodiments, disclosed herein is a probe set for analyzing a biological sample. The probe set may comprise one or more probes from n sets of probes for n target sequences T1, . . . , Tk, . . . . , Tn, in m cycles, wherein: Probe Set 1 comprises P11, . . . , P1j, . . . , and P1m; Probe Set k comprises Pk1, . . . , Pkj, . . . , and Pkm; Probe Set n comprises Pn1, . . . , Pnj, . . . , and Pnm (j, k, m, and n are integers, 2≤j≤m, and 2≤k≤n), where each probe comprises (i) a target hybridization sequence that hybridizes to T1, . . . , Tk, . . . , Tn, respectively, and (ii) an adapter sequence for binding to a nucleic acid origami which comprises a binding staple comprising a staple region that hybridizes to the nucleic acid scaffold and a binding region that directly or indirectly hybridizes to the adapter sequence, and a plurality of fluorescently labelled detection staples. In some embodiments, the biological sample is contacted with Probe Library P11, . . . , Pk1, . . . , and Pn1 in Cycle 1, with Probe Library P1j, Pkj, . . . , and Pnj in Cycle j, and Probe Library P1m, Pkm, . . . , and Pnm in Cycle m. In a particular cycle, the biological sample is contacted with a plurality of nucleic acid origami that hybridize to the probes contacted with the biological sample in the particular cycle, and fluorescent signals from the nucleic acid origami in the biological sample are detected.

In some aspects, the provided methods and nucleic acid origami probes can involve contacting a target nucleic acid with the nucleic acid origami probes. The target nucleic acid can be an endogenous DNA or RNA molecule in the biological sample, or can be comprised by a labelling agent that directly or indirectly binds to an analyte in the biological sample, or can be comprised in a product (e.g., a hybridization product, a ligation product, an extension product (e.g., by a DNA or RNA polymerase), a replication product, a transcription/reverse transcription product, and/or an amplification product such as a rolling circle amplification (RCA) product) of the labelling agent. Particulars of the analytes and/or labelling agents comprising target nucleic acid molecules are described herein, for example in Section II.

In some aspects, provided herein are nucleic acid origami for use in any of the methods disclosed herein. The structure of a DNA origami may be any arbitrary structure as desired. In some embodiments, the method comprises contacting a biological sample with a plurality of nucleic acid origamis, wherein the shape of each origami in the plurality of origami structures is the same, i.e., is not geometrically distinct. In some embodiments, the identity of the target nucleic acid is defined by the binding staple and can be detected via an associated detection staple. Thus, the same core structure can be used for all origamis in the plurality of origamis, and the identity of the target is not tied to the shape of the origami. In some embodiments, the use of origami probes having the same core structure can simplify the probe design and purification protocol. Particulars of the nucleic acid origami probe designs, and the design of additional probes to be used in embodiments of the methods, are described in Section III.

### II. Samples, Analytes, and Target Sequences

A method disclosed herein may be used to process and/or analyze any analyte(s) of interest, for example, for detecting the analyte(s) in situ in a sample of interest. A target nucleic acid sequence for binding a nucleic acid origami disclosed herein may be or be comprised in an analyte (e.g., a nucleic acid analyte, such as genomic DNA, mRNA transcript, or cDNA, or a product thereof, e.g., an extension or amplification product, such as an RCA product) and/or may be or be comprised in a labelling agent for one or more analytes (e.g., a nucleic acid analyte or a non-nucleic acid analyte) in a sample or a product of the labelling agent. Exemplary analytes and labelling agents are described below. In some embodiments, the target nucleic acid sequence is or comprised by an amplification product formed using isothermal amplification or non-isothermal amplification, optionally rolling circle amplification (RCA). In some embodiments, the target nucleic acid sequence is or comprised by a probe or probe set that targets the amplification product. In some embodiments, the target nucleic acid sequence comprises a barcode sequence corresponding to an analyte.

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

(i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

(ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre- permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe (e.g., origami probe) and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

(iv) Embedding

As an alternative to paraffin embedding described above, a biological sample can be embedded in any of a variety of other embedding materials to provide structural substrate to the sample prior to sectioning and other handling steps. In general, the embedding material is removed prior to analysis of tissue sections obtained from the sample. Suitable embedding materials include, but are not limited to, waxes, resins (e.g., methacrylate resins), epoxies, and agar.

In some embodiments, the biological sample can be embedded in a hydrogel matrix. Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method known in the art.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

(v) Staining and Immunohistochemistry (IHC)

To facilitate visualization, biological samples can be stained using a wide variety of stains and staining techniques. In some embodiments, for example, a sample can be stained using any number of stains, and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of destaining or discoloring a biological sample are known in the art, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

(vi) Isometric Expansion

In some embodiments, a biological sample embedded in a hydrogel can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method known in the art. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods are known in the art. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

(ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of methods known to the field (e.g., streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected directly or using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence or origami probe), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer binding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (i.e., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labelling Agent

In some embodiments, provided herein are methods and compositions for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed.

a. Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto). The other molecule can be another endogenous molecule or another labelling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences. In some embodiments, an endogenous analyte and/or a labelling agent (e.g., reporter oligonucleotide attached thereto) can be detected directly or indirectly using a nucleic acid origami as described in Section III. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

b. Ligation

In some embodiments, a product of an endogenous analyte and/or a labelling agent is a ligation product. In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent, for example, the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labelling agent (e.g., the reporter oligonucleotide) or a product thereof.

In some embodiments, the provided methods involve ligating one or more polynucleotides that are part of a hybridization complex that comprises a target nucleic acid for in situ analysis. In some embodiments, the ends of two binding staples can be ligated together. In some embodiments, ligation of the ends of the binding staples stabilizes binding of the nucleic acid origami “padlock” to the target nucleic acid. In some embodiments, binding of a binding staple or adapter may be stabilized by ligation to a probe (e.g., an “anchor” probe) annealed to an adjacent region of the target nucleic acid.

In some embodiments, provided herein is a probe or probe set capable of DNA-templated ligation, such as from a cDNA molecule. See, e.g., U.S. Pat. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Patent No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, a probe such as a padlock probe may be used to analyze a reporter oligonucleotide, which may generated using proximity ligation or be subjected to proximity ligation. In some examples, the reporter oligonucleotide of a labelling agent that specifically recognizes a protein can be analyzed using in situ hybridization (e.g., sequential hybridization) and/or in situ sequencing (e.g., using padlock probes and rolling circle amplification of ligated padlock probes). Further, the reporter oligonucleotide of the labelling agent and/or a complement thereof and/or a product (e.g., a hybridization product, a ligation product, an extension product (e.g., by a DNA or RNA polymerase), a replication product, a transcription/reverse transcription product, and/or an amplification product) thereof can be recognized by another labelling agent and analyzed.

In some embodiments, an analyte (a nucleic acid analyte or non-nucleic acid analyte) can be specifically bound by two labelling agents (e.g., antibodies) each of which is attached to a reporter oligonucleotide (e.g., DNA) that can participate in ligation, replication, and sequence decoding reactions, e.g., using a probe or probe set (e.g. a padlock probe, a SNAIL probe set, a circular probe, or a padlock probe and a connector). In some embodiments, the probe set may comprise two or more probe oligonucleotides, each comprising a region that is complementary to each other. For example, a proximity ligation reaction can include reporter oligonucleotides attached to pairs of antibodies that can be joined by ligation if the antibodies have been brought in proximity to each other, e.g., by binding the same target protein (complex), and the DNA ligation products that form are then used to template PCR amplification, as described for example in Soderberg et al., Methods. (2008), 45(3): 227-32, the entire contents of which are incorporated herein by reference. In some embodiments, a proximity ligation reaction can include reporter oligonucleotides attached to antibodies that each bind to one member of a binding pair or complex, for example, for analyzing a binding between members of the binding pair or complex. For detection of analytes using oligonucleotides in proximity, see, e.g., U.S. Patent Application Publication No. 2002/0051986, the entire contents of which are incorporated herein by reference. In some embodiments, two analytes in proximity can be specifically bound by two labelling agents (e.g., antibodies) each of which is attached to a reporter oligonucleotide (e.g., DNA) that can participate, when in proximity when bound to their respective targets, in ligation, replication, and/or sequence decoding reactions

In some embodiments, one or more reporter oligonucleotides (and optionally one or more other nucleic acid molecules such as a connector) aid in the ligation of the probe. Upon ligation, the probe may form a circularized probe. In some embodiments, one or more suitable probes can be used and ligated, wherein the one or more probes comprise a sequence that is complementary to the one or more reporter oligonucleotides (or portion thereof). The probe may comprise one or more barcode sequences. In some embodiments, the one or more reporter oligonucleotide may serve as a primer for rolling circle amplification (RCA) of the circularized probe. In some embodiments, a nucleic acid other than the one or more reporter oligonucleotide is used as a primer for rolling circle amplification (RCA) of the circularized probe. For example, a nucleic acid capable of hybridizing to the circularized probe at a sequence other than sequence(s) hybridizing to the one or more reporter oligonucleotide can be used as the primer for RCA. In other examples, the primer in a SNAIL probe set is used as the primer for RCA.

In some embodiments, one or more analytes can be specifically bound by two primary antibodies, each of which is in turn recognized by a secondary antibody each attached to a reporter oligonucleotide (e.g., DNA). Each nucleic acid molecule can aid in the ligation of the probe to form a circularized probe. In some instances, the probe can comprise one or more barcode sequences. Further, the reporter oligonucleotide may serve as a primer for rolling circle amplification of the circularized probe. The nucleic acid molecules, circularized probes, and RCA products can be analyzed using any suitable method disclosed herein for in situ analysis.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tm) of the DNA strands. This selectively reduces the concentration of annealed mismatched substrates (expected to have a slightly lower Tm around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

c. Primer Extension and Amplification

In some embodiments, a product here is a primer extension product of an analyte, a labelling agent, a probe or probe set bound to the analyte (e.g., a padlock probe bound to genomic DNA, mRNA, or cDNA), or a probe or probe set bound to the labelling agent (e.g., a padlock probe bound to one or more reporter oligonucleotides from the same or different labelling agents). In some embodiments, nucleic acid origami comprising a nucleic acid scaffold and a binding staple can be contacted with an extension product generated as described herein.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) are known in the art such as linear RCA, a branched RCA, a dendritic RCA, or any combination thereof (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-119, 2000; Faruqi et al, BMC Genomics 2:4, 2000; Nallur et al, Nucl. Acids Res. 29:el 18, 2001; Dean et al. Genome Res. 11 :1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, US 2018/0051322, WO 2017/079406, US 2018/251833, US 2016/0024555, US 2018/0251833 and US 2017/0219465, the contents of each of which are herein incorporated by reference in their entirety. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a portion thereof, where the target analyte is a nucleic acid, or it may be provided or generated as a proxy, or a marker, for the analyte. As noted above, many assays are known for the detection of numerous different analytes, which use a RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe disclosed herein (e.g., a nucleic acid origami probe) may be a hybridization complex formed of a cellular nucleic acid in a sample and an exogenously added nucleic acid probe. The exogenously added nucleic acid probe may comprise an overhang that does not hybridize to the cellular nucleic acid but hybridizes to another probe (e.g., a detectably labelled probe such as a nucleic acid origami probe, or a circularizable probe or probe set). The exogenously added nucleic acid probe may be optionally ligated to a cellular nucleic acid molecule or another exogenous nucleic acid molecule. In other examples, a product comprising a target sequence for a probe disclosed herein (e.g., a nucleic acid origami probe) may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a probe disclosed herein (e.g., a nucleic acid origami probe) may be a probe hybridizing to an RCP. The probe may comprise an overhang that does not hybridize to the RCP but hybridizes to another probe (e.g., a detectably labelled probe such as a nucleic acid origami probe, or HCR or LO-HCR components). The probe may be optionally ligated to a cellular nucleic acid molecule or another probe, e.g., an anchor probe that hybridize to the RCP.

C. Target Sequences

A target sequence for a probe disclosed herein (e.g., a nucleic acid origami probe) may be comprised in any analyte disclose herein, including an endogenous analyte (e.g., a viral or cellular nucleic acid), a labelling agent (e.g., a reporter oligonucleotide attached thereto), or a product of an endogenous analyte and/or a labelling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes (e.g., primary and/or secondary barcode sequences) can be analyzed (e.g., detected or sequenced) using any suitable methods or techniques, including those described herein. In some embodiments, the barcodes can be part of a probe that is used in various methods or techniques such as RNA sequential probing of targets (RNA SPOTs), sequential fluorescent in situ hybridization (seqFISH), single-molecule fluorescent in situ hybridization (smFISH), hybridization-based in situ sequencing (HybISS), multiplexed error-robust fluorescence in situ hybridization (MERFISH), in situ sequencing, targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by hybridization with one or more nucleic acid origami probes disclosed herein. In some embodiments, a nucleic acid origami probe can be used as a detectably labelled probe (e.g., the detection staples of an origami probe are fluorescently labelled for enhanced signal intensity per probe). In some embodiments, a nucleic acid origami probe can be used as an intermediate probe or a scaffold for hybridization of additional probes including detectably labelled probes such as HCR monomers and/or LO-HCR monomers.

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4′ complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and US 2021/0164039 A1, which are hereby incorporated by reference in their entirety.

### III. Analyte Detection Using Nucleic Acid Origami

In some aspects, provided herein are methods comprising in situ assays using microscopy as a readout, e.g., nucleic acid sequencing, hybridization, or other detection or determination methods involving an optical readout, wherein a nucleic acid origami provides a scaffold for binding of one or more detection staples. In some aspects, detection or detection or determination of a sequence of one, two, three, four, five, or more nucleotides of a target nucleic acid is performed in situ in a cell in an intact tissue. In some embodiments, the detection can be used to reveal the presence/absence, distribution, location, amount, level, expression, or activity of the one or more analytes in the sample. In some embodiments, the assay comprises detecting the presence or absence of an amplification product (e.g., RCA product). In some embodiments, the present disclosure provides methods for high-throughput profiling of a large number of targets in situ, such as transcripts and/or DNA loci, e.g., for detecting and/or quantifying nucleic acids and/or proteins in cells, tissues, organs or organisms.

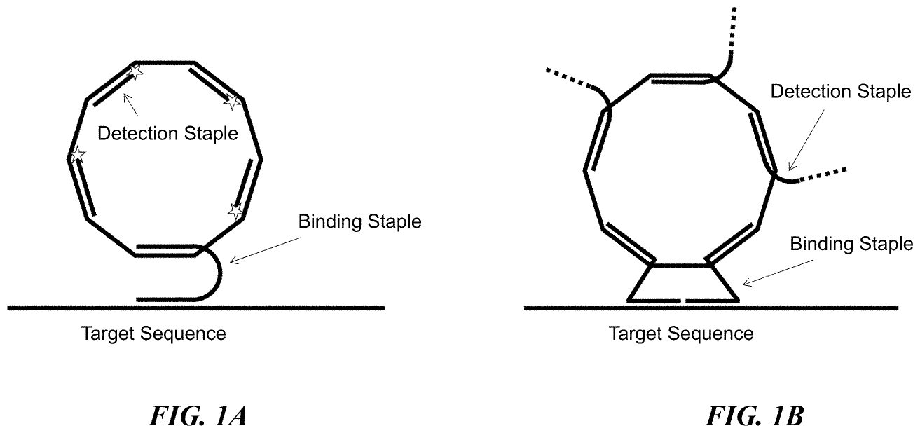

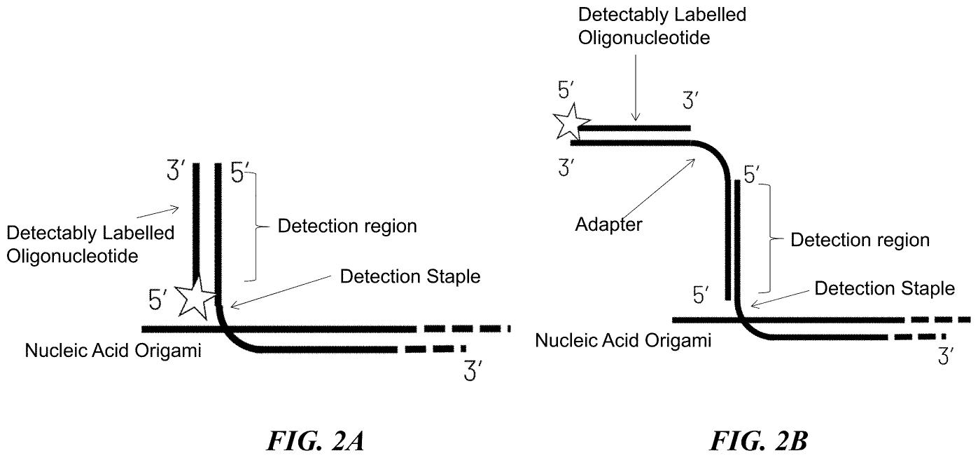

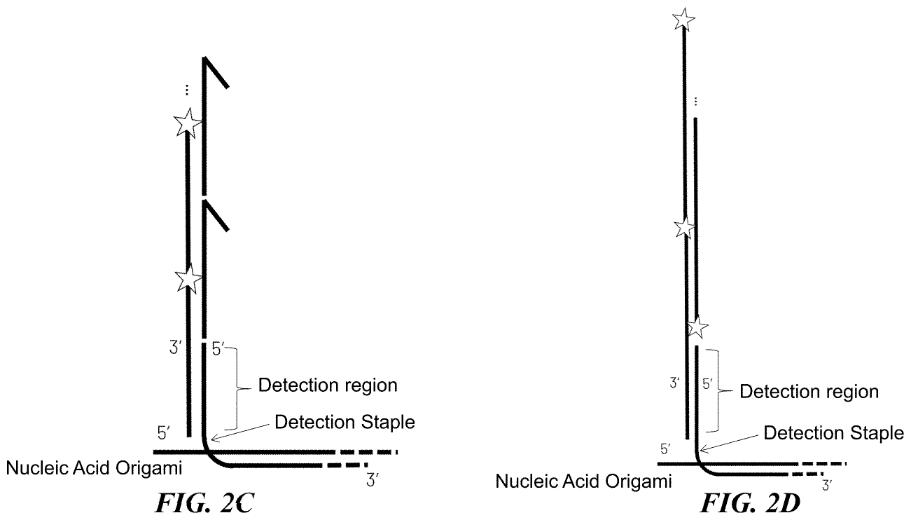

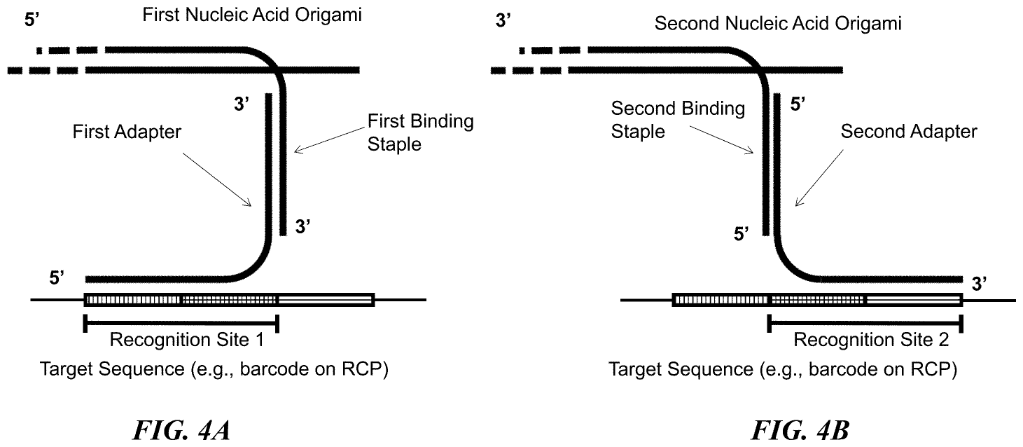

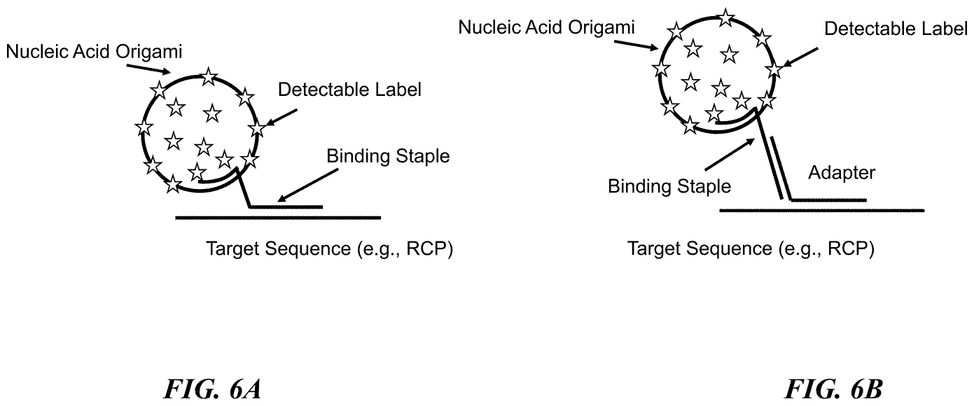

In some aspects, provided herein is a method comprising analyzing biological targets based on in situ hybridization of probes such as nucleic acid origami probes disclosed herein. In some embodiments, the method comprises sequential hybridization of nucleic acid origami (e.g., via a binding staple) to barcoded probes that directly or indirectly bind to biological targets in a sample. In some embodiments, a nucleic acid origami directly binds (e.g., via a binding staple) to one or more probes (e.g., barcoded probes). In some embodiments, a nucleic acid origami indirectly binds to one or more barcoded probes, e.g., via one or more intermediate nucleic acid molecules. In some embodiments, an adapter (e.g., as shown in FIG. 2B, FIGS. 4A-4B, FIG. 6B)) can comprise one or more barcode sequences. In some embodiments, a protruding staple (e.g., a binding staple or a detection staple) of a nucleic acid origami can comprise one or more barcode sequences. In some embodiments, a detection staple (e.g., as shown in FIG. 1B, FIGS. 2A-2D, FIGS. 4A-4B) can comprise one or more barcode sequences. In some embodiments, a binding staple (e.g., as shown in FIGS. 1A-1B, FIGS. 4A-4B, FIG. 5, FIGS. 6A-6B) can comprise one or more barcode sequences.

In some aspects, an in situ hybridization based assay is used to localize and analyze nucleic acid sequences (e.g., a DNA or RNA molecule comprising one or more specific sequences of interest) within a native biological sample, e.g., a portion or section of tissue or a single cell. In some embodiments, the in situ assay is used to analyze the presence, absence, an amount or level of mRNA transcripts (e.g., a transcriptome or a subset thereof, or mRNA molecules of interest) in a biological sample, while preserving spatial context. In some embodiments, the present disclosure provides compositions and methods for in situ hybridization using directly or indirectly labelled molecules, e.g., complementary DNA or RNA or modified nucleic acids, as probes that bind or hybridize to target nucleic acids within a biological sample of interest.

In some embodiments, provided herein is a method comprising DNA in situ hybridization to measure and localize DNA. In some embodiments, provided herein is a method comprising RNA in situ hybridization to measure and localize RNAs (e.g., mRNAs, lncRNAs, and miRNAs) within a biological sample (e.g., a fixed tissue sample). In some embodiments, fluorescently labelled nucleic acid origami probes are hybridized to pre-determined RNA targets, to visualize gene expression in a biological sample. In some embodiments, the method comprises using one or more nucleic acid origami directly or indirectly labelled with a detectable moiety specific to each target. The detectable moiety may produce a fluorescence signal that allows for quantitative measurement of RNA transcripts.

In further embodiments, the nucleic acid origami probes described herein are applied to a multiplexed workflow, wherein consecutive/sequential hybridizations of the nucleic acid origami are used to impart a temporal barcode on target transcripts. Sequential rounds of fluorescence in situ hybridization of nucleic acid origami may be accompanied by imaging and probe stripping, detecting individual transcripts (e.g., RNA transcripts) within a biological sample of interest (e.g., a tissue sample, a single cell, or extracted RNA). In some embodiments, each round of hybridization comprises a pre-defined set of nucleic acid origami probes (e.g., between about 10 and about 50 probes such as 24 to 32 probes) that target unique RNA transcripts. In some examples, the pre-defined set of nucleic acid origami probes is multicolored. In some embodiments, a multiplexed in situ hybridization method using the nucleic acid origami probes described herein may multiplex from lOs to over 10,000 different analytes (e.g., mRNAs), optionally accompanied by imaging, to efficiently and accurately profile the entire transcriptome. In some embodiments, detection of nucleic acid molecules in the sample using the nucleic acid origami probes described herein may be combined with other methods of detection, such as in situ hybridization methods that employ metal tags instead of fluorophores (e.g., imaging mass cytometry). Metal-conjugated antibodies may couple to the metal tags hybridized to transcripts on a biological sample. In some embodiments, mass-cytometry may be used to quantify metal abundances, allowing the concurrent evaluation of RNA and protein within a biological sample.

In some embodiments, the nucleic acid origami probes can be used as readout probes in a multiplexed FISH protocol that is error-robust (e.g., MERFISH). In some embodiments, said protocol comprises non-readout nucleic acid probes (e.g., primary probes) comprising a binding region (e.g., a region that binds to a target such as RNA transcripts) coupled to one or more flanking regions. In some embodiments, each non-readout nucleic acid probe is coupled to two flanking regions. The non-readout nucleic acid probes may hybridize to a transcript (e.g., RNA transcript) within a biological sample (e.g., tissue sample or a single cell), such that florescent readout nucleic acid probes (nucleic acid origami probes) may subsequently serially hybridize to the flanking region(s) of the non-readout nucleic acid probes. In some embodiments, each round of hybridization comprises successive imaging and probe stripping to remove signals from readout nucleic acid probes (nucleic acid origami probes) from previous rounds. RNAs may be imaged by to detect labelled nucleic acid origami probes, and errors accumulated during multiple imaging rounds (e.g., imperfect hybridizations) are detected and/or corrected. In some embodiments, expansion microscopy is employed to increase the number of detected RNA targets without signal overlap. In similar embodiments, non-readout nucleic acid probes are cross-linked to target transcripts prior to imaging. Cross-linking may be performed by any method known in the art. In preferred embodiments, cross-linking is performed using hydrogel tissue embedding. Following said cross-linking steps, barcoding may be performed, comprising sequential hybridizations using readout probes coupled to pre-determined colors to generate unique barcodes (e.g., generating pseudo colors from consecutive hybridizations).

In some embodiments, one or more barcodes of a probe are targeted by detectably labelled nucleic acid origami probes. In some embodiments, one or more decoding schemes are used to decode the signals, such as fluorescence, for sequence determination. In some embodiments, the methods provided herein comprise analyzing the barcodes by sequential hybridization and detection with a plurality of nucleic acid origami. Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); U.S. Pat. No. 10,457,980 B2; US 2016/0369329 A1; US 2021/0017587A1; and US 2017/0220733 A1, all of which are hereby incorporated by reference in their entirety. In some embodiments, these assays enable signal amplification, combinatorial decoding, and error correction schemes at the same time.

Similar strategies of in situ hybridization using variations of FISH techniques may also be adopted by methods described herein. In some embodiments, a method comprises non-barcoding multiplexed FISH protocols (e.g., ouroboros sm-FISH (osmFISH)), wherein the nucleic acid origami probes described herein are used as readout probes. Non-barcoding methods may be limited to detecting a specific number of targets, defined by the number of hybridization rounds performed. In some embodiments, imaging is performed following each hybridization round, wherein the nucleic acid origami probe is stripped after imaging, allowing for subsequent hybridization and imaging rounds. In some aspects, a nucleic acid origami structure described herein is used as a primary probe, and the method can comprise sequentially hybridizing and removing probes associated with a signal or with the absence of signal (e.g., for sequential signal code sequences comprising a “dark” cycle) to the nucleic acid origami. For example, in some embodiments, the nucleic acid origami comprises detection staples having protruding detection regions, wherein the detection region can comprise a barcode sequence or be linked to a barcode sequence in an adapter for sequential decoding hybridizations (e.g., as shown in FIGS. 9 and 10). Exemplary methods for sequential decoding of barcode sequences have been described, for example, in US20210340618, the content of which is herein incorporated by reference in its entirety. Thus, the nucleic acid origami can function as a primary probe hybridized to a nucleic acid analyte or labelling agent (e.g., a labelling agent comprising a reporter oligonucleotide) in the sample.

In some embodiments, provided herein is a method comprising linking sequencing information and spatial information of targets within endogenous environments. For example, analysis of nucleic acid sequences may be performed directly on DNA or RNA within an intact biological sample of interest, e.g., by in situ sequencing. In some embodiments, the present disclosure allows for the simultaneous identification and quantification of a plurality of targets, such as 100s, 1000s, or more of transcripts (e.g., mRNA transcripts), in addition to spatial resolution of said transcripts. In some aspects, the spatial resolution of transcripts may be subcellular. Optionally, the spatial resolution may be increased using signal amplification strategies described herein.

In some embodiments, fluorescent dyes are used to target nucleic acid bases, and padlock probes are used to target RNAs of interest in situ. In some embodiments, mRNAs are reverse transcribed into cDNAs, and padlock probes are able to bind or couple to cDNAs. In some embodiments, padlock probes comprise oligonucleotides with ends that are complementary to a target sequence (e.g., target cDNA transcripts). Upon hybridization of padlock probes to the target sequence, enzymes may be used to ligate the ends of the padlock probes, and catalyze the formation of circularized DNA.

In some embodiments, the ends of the padlock probes are in close proximity upon hybridization to the target RNA or cDNA, to allow ligation and circularization of the padlock probe. The padlock probes may additionally comprise one or more barcode sequences. In alternative embodiments, there may be a gap between the ends of the padlock probes upon hybridization to the target RNA or cDNA, that must be filled with nucleic acids (e.g., by DNA polymerization), prior to ligation of the ends of the padlock probes and circularization. In some embodiments, the gap between to ends of the padlock probes is of variable length, e.g., up to four base pairs, and can allow reading out the actual RNA or cDNA sequence. In some embodiments, the DNA polymerase has strand displacement activity. In some embodiments, the DNA polymerase may instead not have strand displacement activity, such as the polymerase used in barcode in situ target sequencing (BaristaSeq) which provides read-length of up to 15 bases using a gap-filling padlock probe approach. See, e.g., Chen et al., Nucleic Acids Res. 2018, 46, e22, incorporated herein by reference in its entirety.

A method described herein may comprise DNA circularization and amplification (e.g., rolling circle amplification), at the location of padlock probes. In some embodiments, amplification results in multiple repeats of padlock probe sequences. The padlock probe sequences can be detected using hybridization of the nucleic acid origami probes described herein. In some embodiments, amplicons are stabilized by crossing-linking described herein, during the sequencing process. In some embodiments, the in situ sequencing methods presented in this disclosure may be automated on a microfluidic platform. In some instances, the products of amplification comprising one or more barcodes of a probe are targeted by detectably labelled nucleic acid origami probes.

In some embodiments, the methods described herein comprise performing in situ sequencing of a sequence (e.g., a barcode sequence) in the detection region of one or more detection staples of a nucleic acid origami, or in an adapter associated with said detection region. In some embodiments, in situ sequencing involves incorporation of a labelled nucleotide (e.g., fluorescently labelled mononucleotides or dinucleotides) in a sequential, template-dependent manner or hybridization of a labelled primer (e.g., a labelled random hexamer) to a nucleic acid template such that the identities (e.g., nucleotide sequence) of the incorporated nucleotides or labelled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363, which are hereby incorporated by reference in their entirety. In addition, examples of methods and systems for performing in situ sequencing are described in US 2019/0177718, US 2019/0194709, and in U.S. Pat. Nos. 10,138,509, 10,494,662, and 10,179,932, which are hereby incorporated by reference in their entirety. Exemplary decoding techniques for in situ sequencing comprise, but are not limited to, STARmap (described for example in Wang et al., (2018) Science, 361(6499) 5691), MERFISH (described for example in Moffitt, (2016) Methods in Enzymology, 572, 1-49), and FISSEQ (described for example in US 2019/0032121), which are hereby incorporated by reference in their entirety. In some embodiments, methods can comprise sequentially contacting the sample with nucleic acid origami associated with different detectable labels (or the absence of a label) in order to decode a sequence of interest, wherein the binding region of a binding staple hybridizes to the sequence of interest. In some instances, detectably labelled nucleic acid origami probes can be applied to detect probes or products generated in the provided exemplary systems for performing in situ sequencing.

Probes comprising nucleic acid origami may be used in any suitable in situ hybridization or sequencing assay disclosed herein, for example, for signal enhancement (e.g., as disclosed in Section III-B) and/or for providing a scaffold for complex formation (e.g., as in HCR or LO-HCR) for analyte detection and/or decoding.

A. Nucleic Acid Origami

In some embodiments, disclosed herein are nucleic acid origami structure(s) which may be used as a probe or a probe set for analyzing molecules or complexes thereof in situ in a sample. In some embodiments, a nucleic acid origami (e.g., a DNA origami) disclosed herein is a two- or three-dimensional arbitrary shape formed, typically at the nanoscale, from one or more molecules comprising nucleic acid sequence(s) capable of Watson-Crick base pairing. Nucleic acid origami formation may involve the folding of one or more long single-stranded nucleic acid molecules aided by multiple smaller “staples,” which bind to specific regions of the longer strand (the “template” or “scaffold”). In some embodiments, one or more template or scaffold molecules and/or one or more staple molecules are designed and synthesized de novo. In some embodiments, one or more templates and various staples are mixed, and the mixture is heated and cooled. As the mixture cools, the staples can help pull the template(s) into a pre-defined two- or three-dimensional design, which may be observed via several methods, including electron microscopy, atomic force microscopy, or fluorescence microscopy. In some aspects, the origami structure can be any suitable shape and size. For example, if desired, a larger origami structure made be used by increasing the size of the scaffold. In some cases, if desired, an origami shape that is efficient in terms of surface area may be used.

In some embodiments, a nucleic acid origami disclosed herein comprises one or more non-naturally occurring nucleic acid nanostructures. In some embodiments, the nucleic acid nanostructure comprises one or more two-dimensional arbitrary shapes (e.g., a flat sheet). In some embodiments, the nucleic acid nanostructure comprises one or more three-dimensional arbitrary shapes. In some embodiments, the non-naturally occurring nucleic acid nanostructure is formed by folding a single stranded nucleic acid scaffold into a custom shape and using staples (e.g., oligonucleotide strands) to hybridize with the folded single stranded nucleic acid scaffold and hold it into the custom shape. The structure of a nucleic acid origami may be any arbitrary structure as desired.

In some embodiments, a nucleic acid origami is a megadalton-scale nanostructure created from a plurality of DNA strands. According to an exemplary aspect, a DNA origami is created from a scaffold strand of DNA, which is arranged into a desired macromolecular object of a custom shape. Staples strands of DNA, which may be shorter than the scaffold strand of DNA, can be used to direct the folding or other orientation of the scaffold strand of DNA into a programmed arrangement. One or more strands of DNA may be folded or otherwise positioned into a desired structure or shape, which may then be secured into a desired shape or structure by one or more other strands of DNA, such as a plurality of staple strands of DNA. Exemplary methods of making nucleic acid origami such as DNA origami structures of arbitrary design or desired design are described in Rothemund, “Folding DNA to Create Nanoscale Shapes and Patterns,” Nature March 2006, p. 297-302, vol. 440; Rothemund, “Design of DNA Origami,” Proceedings of the International Conference of Computer-Aided Design (ICCAD) 2005; U.S. Pat. No. 7,842,793; Douglas et al., Nuc. Acids Res., vol. 37, no. 15, pp. 5001-5006; Douglas et al., Nature, 459, pp. 414-418 (2009); Andersen et al., Nature, 459, pp. 73-76 (2009); Deitz et al., Science, 325, pp. 725-730 (2009); Han et al., Science, 332, pp. 342-346 (2011); Liu et al., Angew. Chem. Int. Ed., 50, pp. 264-267 (2011); Zhao et al., Nano Lett., 11, pp. 2997-3002 (2011); Woo et al., Nat. Chem. 3, pp. 620-627 (2011); and Torring et al., Chem. Soc. Rev. 40, pp. 5636-5646 (2011), each of which is incorporated herein by reference in its entirety.

The structure of a DNA origami may be any arbitrary structure as desired. In some embodiments, the method comprises contacting a biological sample with a plurality of nucleic acid origamis, wherein the shape of each origami in the plurality of origamis is the same, i.e., is not geometrically distinct. In some embodiments, the identity of the target nucleic acid is defined by the binding staple associated with the origami and can be detected via one or more detection staples associated with the origami. Thus, the same core structure can be used for all origamis in the plurality of origamis, and the identity of the target is not tied to the shape of the origami. In some instances, the same nucleic acid scaffold can be designed with different staple sequences to generate various shapes and such shapes generate sufficient binding sites for be detection via one or more detection staples. In some aspects, the identity of the target is not associated with the shape of the origami, for example by using spatially distinct nucleic acid structures, geometrically distinct nucleic acid structures, spatially resolvable nucleic acid structures, or spatially observable nucleic acid structures. In some embodiments, the observable signal from a detectably labelled oligonucleotide or probe that directly or indirectly binds the nucleic acid origami provides the information associated with the target nucleic acid rather than a shape or geometric property of the nucleic acid structure used (e.g., of the origami). In some cases, the shape or geometric property of the origami is not utilized as a distinguishing feature of the origami. In some cases, the origami is used for amplifying signals.

It is to be understood that the present disclosure does not rely on any particular method of making nucleic acid origami or any particular two- or three-dimensional shape. A plurality of nucleic acid origami structures each having a unique shape may be used to barcode or otherwise identify specific nucleic acids or nucleic acid sequences, but such use of nucleic acid origami shapes/structures as barcodes or identifiers is not essential. It is to be further understood that aspects of the ability to design nucleic acid origami with desired hybridization sites, staples, or desired probes (e.g., probes that link a nucleic acid origami to a target sequence, and/or probes that comprise barcode sequences) is useful to barcode or otherwise identify specific nucleic acids or nucleic acid sequences.

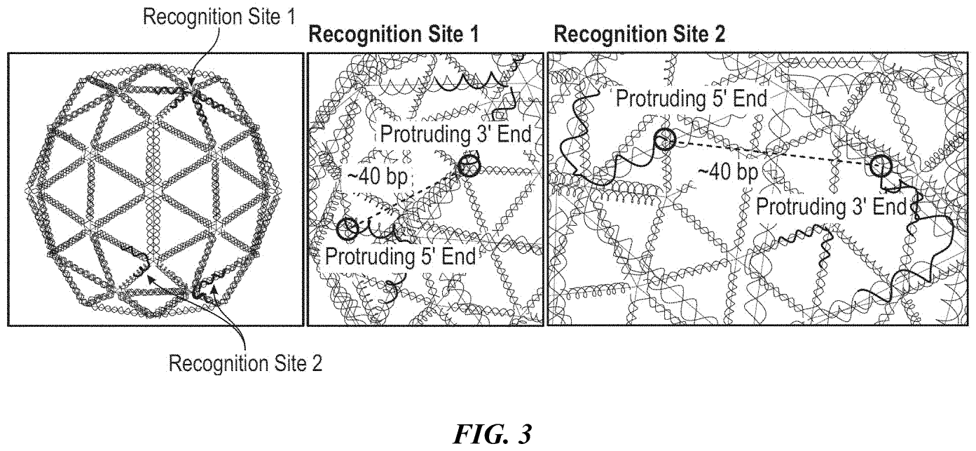

In some embodiments, the nucleic acid origami is a polyhedral mesh or wireframe structure. Methods of generating polyhedral mesh nucleic acid origami structures have been described, for example, in Benson et al., Nature 523, 441-444 (2015), the content of which is herein by reference in its entirety. Software and design pipelines for origami structures such as polyhedral mesh or other structures have also been described by Benson et al. 2015, for example. In some embodiments, a suitable nucleic acid origami can be designed starting from a 3D mesh representation of the target structure with a goal of replacing each of the incorporating edges by a rigid DNA double helix, thus in effect rendering the proposed geometry in DNA. In some embodiments, the mesh structure of the nucleic acid origami is fully triangulated, providing structural rigidity to convex polyhedra (e.g., spherical polyhedral meshes). In some embodiments, the nucleic acid origami does not require stabilization from multivalent cations or high concentrations of monovalent cations. The nucleic acid origami structures provided herein can remain stable in physiological buffers, such as phosphate buffered saline (PBS) and Dulbecco's Modified Eagle Medium (DMEM). In some embodiments, the nucleic acid origami structures provided herein can be folded in physiological buffers. In some embodiments, the nucleic acid origami is a spherical polyhedral mesh (e.g., as shown in FIG. 3).

In some embodiments, a nucleic acid scaffold strand is routed to form an origami structure (e.g., a polyhedral mesh). The routing of the staple strands (DNA oligonucleotides that at least in part help to hold the structure in place) can then follow implicitly from the routed nucleic acid scaffold. Staples can be used to complete the edge connections at the vertices. Although the routing of the staple strands may be fully determined by the scaffold routing, the placement of staple-strand breakpoints can be freely modified.

In some embodiments, the number of staples in the origami structure can be determined by the routing of the staple strands (e.g., by the number of edge connections at vertices). In some embodiments, the nucleic acid origami comprises a nucleic acid scaffold and between 50 and 250 staples (e.g., between 100 and 200 staples, or between 100 and 150 staples). In the case of an exemplary nucleic acid origami described in Example 1, the spherical wireframe origami structure has 132 staples. In some embodiments, staples (or staple regions of binding or detections staples, as described below) can be between about 15 and 60 nucleotides in length, e.g., between about 20 and 50 nucleotides, between about 20 and 40 nucleotides in length, or between about 30 and 40 nucleotides in length.

In some embodiments, one or more staples for an origami can be modified for various suitable uses, such as to hybridize to other nucleic acids (e.g., a target nucleic acid or for detection). In some embodiments, a modified staple (e.g., a binding staple or detection staple) is modified to gain additional function (e.g., provide a binding site for a complementary oligonucleotide) and retains function as a staple (e.g., for structure of the core origami). In some embodiments, a staple design is modified to further comprise a region protruding from the folded core of the nucleic acid origami (i.e., a region that does not bind to the nucleic acid scaffold). In some embodiments, a plurality of staples for an origami can be modified to comprise a region protruding from the folded core of the nucleic acid origami (e.g., to provide a binding site for a complementary oligonucleotide). In some embodiments, the protruding region of the nucleic acid origami staple is between about 10 and about 60 nucleotides in length, optionally between about 15 and about 35, e.g., about 20, nucleotides in length. In some embodiments, the protruding region of the staple is shorter than the non-protruding region of the staple (e.g., the binding region of a binding staple is shorter than the staple region of the binding staple, and/or the detection region of a detection staple is shorter than the staple region of the detection staple). In some embodiments, the length of the staple region of a modified staple (e.g., a detection or binding staple) is equal to or longer than the protruding region of the staple. In some embodiments, the staple region of a modified nucleic acid origami staple is between or between about any of 20 and 65, 20 and 60, 30 and 60, 40 and 60, 50 and 60, 20 and 50, 30 and 50, or 40 and 50 nucleotides in length. In some embodiments, the staple region of a modified nucleic acid origami is between about 40 and 60 nucleotides in length.

In some embodiments, at least one staple for an origami is modified to further comprise a region protruding from the folded core of the nucleic acid origami (e.g., a binding region or a detection region). In some embodiments, at least 1%, 2%, 5%, 10%, 15%, 20%, 25%, 30%, 40%, 50%, 60%, 70%, 80%, or 90% of the staples that contribute to the structure of the core origami are modified to further comprise a region protruding from the folded core of the nucleic acid origami. In some embodiments, at least about 20% or 25% of the staples that contribute to the structure of the core origami are modified to further comprise a region protruding from the folded core of the nucleic acid origami or to be directly or indirectly linked to a detectable moiety (e.g., a fluorescent label).

In some embodiments, the nucleic acid origami comprises a binding staple, wherein the binding staple comprises a sequence complementary to the nucleic acid origami (the staple region), and a binding region protruding from the folded core, wherein the binding region directly or indirectly (e.g., via an adapter) hybridizes to a nucleic acid molecule. The binding region may be linked to the staple region via a linker. In some embodiments, the binding region is between about 10 and about 50 nucleotides in length, optionally between about 15 and about 25, e.g., about 20, nucleotides in length. Optionally, the linker can be between about 1 and about 10 nucleotides in length, optionally between about 2 and about 5 nucleotides in length.

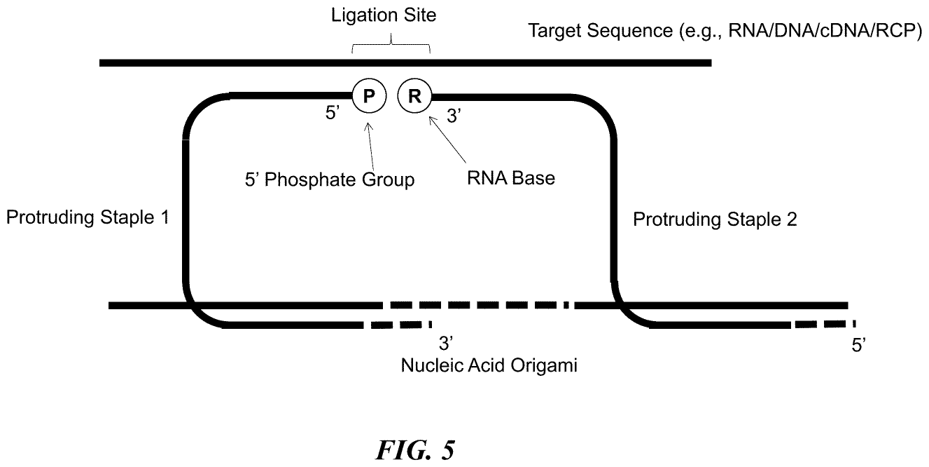

In some embodiments (e.g., as described in subsection C, “Detection Design”), the nucleic acid origami comprises a plurality of binding staples, optionally wherein the binding region of a first binding staple comprises a 5′ end of the first binding staple, and the binding region of a second binding staple comprises a 3′ end of the second binding staple. In some embodiments, the protruding ends of the two binding staples are separated by a distance of no more than twice the length of the protruding strands. In some embodiments, the protruding ends of the two binding staples are separated by between about 30 and 60 nucleotides in the nucleic acid origami (e.g., by no more than about 40 bp, as shown in FIG. 3, for example, wherein the length of each of the protruding ends of the binding staples is about 20). In some aspects, the location where the binding staples bind in the origami may be different depending on the shape of the origami. In some examples, the protruding ends of the two binding staples are separated by a distance that allows the protruding regions of the two binding staples to hybridize to a target nucleic acid and allow their ends to be in sufficient proximity to be ligated when hybridized. In some embodiments, the binding regions of two binding staples can hybridize to a target nucleic acid such that their ends can be ligated in a template ligation reaction (e.g., FIG. 5 and as described further in subsection C, “Detection Design”).

In some embodiments, the nucleic acid origami comprises a plurality of detection staples, wherein each detection staple is directly or indirectly labelled with a detectable moiety. In some embodiments, a detection staple is directly coupled (e.g., covalently coupled) to a detectable moiety. In some embodiments, the detection staple does not comprise a protruding region (e.g., does not comprise a region that does not hybridize to the nucleic acid origami). In other embodiments, the detection staple comprises a region protruding from the folded core, similarly to the binding region of a binding staple. The protruding region of a detection staple can comprise a detection region and optionally a linker region. In some embodiments, the detection region is between about 10 and about 50 nucleotides in length, optionally between about 15 and about 25, e.g., about 20, nucleotides in length. Optionally, the linker can be between about 1 and about 10 nucleotides in length, optionally between about 2 and about 5 nucleotides in length.

In some embodiments, the nucleic acid origami structure is designed for signal enhancement of rolling circle products (RCP), as shown in FIG. 1A, or for target nucleic acid molecule detection, as shown in FIG. 1B. In some embodiments, the target nucleic acid is a product or derivative of the analyte. In some embodiments, the target nucleic acid is generated using a probe bound to the analyte (e.g., RCP generated using a primary probe such as a circularized probe as template, where the probe is bound to a nucleic acid analyte of interest). In some embodiments, a spherical wireframe DNA origami structure (Benson et al., Nature 523, 441-444 (2015) can be modified.

Without being bound by theory, the nucleic acid origami signal enhancement structures as shown in FIG. 1A allow for the enhancement of the signal of a target nucleic acid by a theoretical n-fold, wherein n is the number of detectably labelled detection staples, as the nucleic acid origami occupies the binding site of the original fluorescent oligo in a conventional RCP detection assay, effectively replacing a single detectable moiety with n-detectable oligos (e.g., fluorescent oligos). In this way, the structure can be used as a signal amplifier. This can be particularly useful as it potentially means that RCPs can be detected on a 10× or even 5× objective which would speed up imaging time dramatically. Furthermore, although FIG. 1A depicts a nucleic acid molecule comprising detection staples that are directly labelled with a detectable moiety, in other embodiments, detection staples comprising protruding ends can be used in a nucleic acid origami for signal enhancement of an RCP. The protruding detection regions of the detection staples may be designed to be compatible with existing methods such as hybridization chain reaction or variations thereof (e.g., LO-HCR or RCA-HCR) to boost signal intensity further.

In some embodiments, the nucleic acid origami design as shown in FIG. 1B allows detection of a target nucleic acid (e.g., mRNA or derivative thereof) without requiring amplification. As shown in FIG. 1B, the nucleic acid origami can be designed with two binding staples that anneal to adjacent regions of the target nucleic acid such that the ends of the two binding staples can be optionally ligated by template ligation (an “origami-padlock”). The approach taken by the origami-padlock allows for the detection of mRNA directly using only T4 RNA ligase 2 without continuing with the Phi29 amplification step. This further saves in costs for enzymes as well as additional QC that will need to be run and makes the method faster as well as more efficient. Techniques that do not require amplification, such as smFISH or MERFISH, typically require 30-50 different fluorescent probes to target the mRNA. In contrast, as shown in Example 5, the methods provided herein can be performed using 4 or fewer origami-padlocks to detect a target nucleic acid, and do not require amplification.

In some embodiments, the detection staple is covalently or non-covalently linked to the detectable moiety. In some embodiments, the 3′ end and/or the 5′ end of the detection staple is linked to a molecule of the detectable moiety. As shown in FIGS. 2A-2B, in some embodiments, the detection staple comprises a detection region protruding from the folded core. The detection region can comprise the 5′ end of the detection staple, as shown in FIGS. 2A-2B, or the detection region can comprise the 3′ end of the detection staple. In some embodiments, the detection region directly hybridizes to a detectably labelled oligonucleotide, as shown in FIG. 2A. In other embodiments, the detection region directly hybridizes to an adapter which directly hybridizes to a detectably labelled oligonucleotide, as shown in FIG. 2B. In some embodiments, the adapter comprises a toehold region that does not hybridize to the detection strand or the detectably labelled oligonucleotide.

In some embodiments, the detectably labelled oligonucleotide is directly or indirectly (e.g., via an adapter) bound to the nucleic acid origami before the contacting step. In some embodiments, the detectably labelled oligonucleotide is not pre-bound to the nucleic acid origami prior to the contacting step, and the method further comprises contacting the sample with the detectably labelled oligonucleotide and/or the adapter oligonucleotide. In other embodiments, as shown in FIG. 1A, the detection staple does not comprise a detection region protruding from the folded core, and the detectable moiety is linked to a sequence of the detection staple that binds to the folded core. In some embodiments, the step of detecting the nucleic acid origami in the biological sample comprises detecting a signal from the detectably labelled oligonucleotide or the detectable moiety. In some embodiments, the nucleic acid origami comprises a plurality of detection staples, optionally wherein the nucleic acid origami comprises at least 5, at least 10, at least 15, at least 20, at least 25, at least 30, at least 35, at least 40, at least 45, or at least 50 detection staples.

In some embodiments, the protruding detection region of the detection staple provides a platform for assembly of a hybridization complex for signal amplification such as via a hybridization chain reaction (HCR), as shown in FIGS. 2C-2D. In some embodiments, the detecting step comprises: i) contacting the biological sample with a plurality of hybridization chain reaction (HCR) units, wherein: one or more HCR units of the plurality are detectably labelled, the detection region comprises or is linked to an HCR initiator sequence that hybridizes to an HCR unit of the plurality to initiate an HCR, and an HCR complex comprising the one or more detectably labelled HCR units is generated; and ii) detecting a signal from the HCR complex bound to the nucleic acid molecule in the biological sample, thereby analyzing the biological sample.

In some embodiments, the protruding detection region comprises a hybridization sequence (e.g., for hybridizing to a detectably labelled oligonucleotide, an adapter, or a unit of a hybridization complex e.g., an HCR unit) and a linker linking the hybridization sequence to a sequence of the detection staple that binds to the folded core. In some instances, each scaffold comprises a plurality of assembled hybridization complex for signal amplification (e.g., HCR). In some embodiments the hybridization sequence is between about 10 and about 50 nucleotides in length, optionally between about 15 and about 25, e.g., about 20, nucleotides in length. In some embodiments, the linker sequence is between about 1 and about 10 nucleotides in length, optionally between about 1 and about 5 nucleotides in length, e.g., about 2 nucleotides in length. For the signal enhancement design, this hybridization sequence is complementary to specific detectably labelled oligonucleotides (FIGS. 2A-2B). For the nucleic acid targeting design, the hybridization sequence can either be complementary to a specific oligonucleotide that will act as the initiator for the LO-HCR chemistry, or be designed to act as an initiator for the HCR hairpins (FIGS. 2C-2D). In some embodiments, the assembly of a hybridization complex for signal amplification (e.g., HCR or LO-HCR) is a linear assembly or a non-linear assembly, e.g., a branched HCR. In some embodiments, the assembled hybridization complex for signal amplification (e.g., HCR) is in one dimension or in multiple dimensions. In some embodiments, the plurality of units of the hybridization complex for signal amplification (e.g., HCR units) comprise one or more linear nucleic acid molecules (e.g., as shown in FIG. 2C) and/or one or more hairpin nucleic acid molecules (e.g., as shown in FIG. 2D). In some embodiments, the plurality of units of the hybridization complex for signal amplification (e.g., HCR units) comprise one or more units that are not detectably labelled. In some embodiments, the one or more non-detectably labelled units of the hybridization complex serve as a splint that hybridizes to two or more detectably labelled units (e.g., HCR units). In some embodiments, the one or more non-detectably labelled units (e.g., HCR units) comprise a toehold region (e.g., as shown in FIG. 2C) that does not hybridize to the detectably labelled units. In some cases, the detection region comprises the initiator sequence for the assembly of the hybridization complex for signal amplification (e.g., HCR initiator). In some embodiments, the detection region hybridizes to an adapter which hybridizes to an initiator comprising the initiator sequence. In some embodiments, the adapter comprises a toehold region that does not hybridize to the detection strand or the initiator (e.g., HCR initiator). In some embodiments, the toehold region is between about 5 and about 20 nucleotides in length, e.g., about 10 nucleotides in length. In some embodiments, adapter and/or the initiator hybridized thereto is dissociated from the detection strand in the absence of a denaturing agent, e.g., formamide. In some embodiments, the dissociation comprises contacting the biological sample with a nucleic acid that hybridizes to the toehold region and displaces the adapter from the detection strand.

FIG. 3 shows an exemplary nucleic acid origami structure comprising four binding staples for binding to a target nucleic acid (e.g., a RCP, an intermediate probe or a nucleic acid molecule of the cell). In the origami design shown in FIG. 3, the binding staples are positioned in pairs in Recognition Site 1 or Recognition Site 2. In each of the two sites, one of the protruding staples protrudes from the structure at its 5′ end and the other strand protrudes from its 3′ end. The protruding end of a binding staple is referred to as a “binding region.” In some embodiments, the binding region comprises a hybridization sequence (e.g., for hybridizing to a target nucleic acid or an adapter) and a linker linking the hybridization sequence to a sequence of the binding staple that binds to the folded core. In some embodiments the hybridization sequence is between about 10 and about 50 nucleotides in length, optionally between about 15 and about 25, e.g., about 20, nucleotides in length. In some embodiments, the linker sequence is between about 1 and about 10 nucleotides in length, optionally between about 1 and about 5 nucleotides in length, e.g., about 2 nucleotides in length. The different polarities (i.e., the binding staple protruding from the 3′ end or the 5′ end) of the binding staples within the design shown in FIG. 3 design make the structure compatible with a 2-site detection probe displacement (“2-LSD”) design, as shown in FIGS. 4A-4B. According to the 2-LSD design, the target nucleic acid comprises a nucleotide barcode sequence, wherein the nucleotide barcode sequence corresponds to a specific signal code sequence that may be derived by interrogating the nucleotide barcode sequence with sequential detection probes (e.g., nucleic acid origami probes), each yielding a signal. The signals together make up a signal code sequence. In some embodiments, the nucleotide barcode sequence comprises at least two adjacent barcode positions, each barcode position comprising a barcode subunit pair comprising a first barcode subunit and a second barcode subunit (Recognition Site 1 and Recognition Site 2 in FIGS. 4A-4B), wherein the second barcode subunit from each barcode position at least partially overlaps with the first barcode subunit of the adjacent barcode position in the sequence. The strand protruding from the 3′ end is able to bind the adapter probes binding to the R1 site on the RCP (FIG. 4A), the strand protruding from the 5′ end is able to bind the adapter probes recognizing the R2 site on the RCP (FIG. 4B). This negates the need to have two separate structures to recognize adapter probes on either site on the RCP, thereby allowing the use of the same nucleic acid origami structure in multiple sequencing cycles.

In some embodiments, the nucleic acid origami structure can be used to directly detect a nucleic acid molecule, e.g., an mRNA molecule. As shown in FIG. 5, in some embodiments, the nucleic acid origami comprises a binding staple with a protruding 5′ end and a binding staple with a protruding 3′ end. In some embodiments, the 5′ protruding binding staple is designed to have a phosphate group at its 5′ end and the 3′ protruding binding staple has an RNA base at its 3′ end. The two protruding staples hybridize to the RNA in such a fashion as to position the 5′ phosphate group next to the 3′ RNA base of the other staple strand. In some embodiments, a plurality (e.g., four) of DNA origami structures are designed per gene, each targeting a specific region of the mRNA molecule.

As discussed above, in some embodiments, ligation of the ends of the two binding staples can stabilize the position of a nucleic acid origami on a target molecule. However, in other embodiments, the nucleic acid origami can directly or indirectly bind to the target nucleic acid via a single binding staple, or via two or more binding staples that are not ligated. In some embodiments, a binding region of a binding staple can hybridize adjacent to an anchor probe, wherein the end of the binding staple and the adjacent end of the anchor probe can be ligated to stabilize hybridization of the binding staple to the target nucleic acid.

In some embodiments, the detectably labelled oligonucleotide is directly or indirectly (e.g., via an adapter) bound to the nucleic acid origami before the contacting step. In some embodiments, the detectably labelled oligonucleotide is not pre-bound to the nucleic acid origami prior to the contacting step, and the method further comprises contacting the sample with the detectably labelled oligonucleotide and/or the adapter oligonucleotide. In other embodiments, as shown in FIG. 1A, the detection staple does not comprise a detection region protruding from the folded core, and the detectable moiety is linked to a sequence of the detection staple that binds to the folded core. In some embodiments, the step of detecting the nucleic acid origami in the biological sample comprises detecting a signal from the detectably labelled oligonucleotide or the detectable moiety. In some embodiments, the nucleic acid origami comprises a plurality of detection staples, optionally wherein the nucleic acid origami comprises at least 5, at least 10, at least 15, at least 20, at least 25, at least 30, at least 35, at least 40, at least 45, or at least 50 detection staples.

In some embodiments, the protruding detection region of the detection staple provides a platform for assembly of a hybridization complex for signal amplification, e.g., a hybridization chain reaction (HCR) or linear-oligo hybridization chain reaction (LO-HCR), as shown in FIGS. 2C-2D. Methods of HCR and LO-HCR are further described in the “Hybridization Complexes and Methods for Signal Amplification” section below.

In some embodiments, the method does not include successive ligation of various origami structures or polynucleotides attached thereto. In some embodiments, the method does not include successive ligation of multiple origami structures of various shapes (e.g., geometrically distinct) to the analyte or a derivative thereof. In some embodiments, the method does not include ligation at multiple positions corresponding to the sequence of the target analyte to build a sequence of the analyte. In some embodiments, the ligation of two polynucleotides is performed (e.g., of a first and second protruding staple) wherein both polynucleotides are hybridized to the same origami structure (e.g., are a part of the same origami). In some embodiments, the origami binds directly or indirectly to a target nucleic acid (or a derivative thereof such as an amplification product or complementary sequence) but the origami structure is not ligated to the target nucleic acid.

B. Signal Enhancement Design

In some aspects provided herein, the nucleic acid origami structure is designed for signal enhancement of a barcode nucleotide sequence, such as barcoded rolling circle products (RCP), as shown in FIG. 1A. Without being bound by theory, the nucleic acid origami signal enhancement structures as shown in FIG. 1A allow for the enhancement of the signal of a target nucleic acid by a theoretical n-fold, wherein n is the number of detectably labelled detection staples, as the nucleic acid origami occupies the binding site of the original fluorescent oligo in a conventional RCP detection assay, effectively replacing a single detectable moiety with n-detectable oligos (e.g., fluorescent oligos). In this way, the structure can be used as a signal amplifier. This can be particularly useful as it potentially means that RCPs can be detected on a 10× or even 5× objective which would speed up imaging time dramatically. Furthermore, although FIG. lA depicts a nucleic acid molecule comprising detection staples that are directly labelled with a detectable moiety, in other embodiments, detection staples comprising protruding ends can be used in a nucleic acid origami for signal enhancement of an RCP. The protruding detection regions of the detection staples may be designed to be compatible with existing methods such as hybridization chain reaction or variations thereof (e.g., LO-HCR or RCA-HCR) to boost signal intensity further.

In some embodiments, the nucleic acid origami is used as the “reporter” probe according to decoding barcode sequences and/or 2-LSD methods described above. Thus, the decoding probe that binds to a nucleotide barcode subunit can be the binding region of a binding staple, or an adapter that binds to the binding region of a binding staple. As shown in FIG. 4A, a first binding staple can comprise a 3′ protruding binding region that binds to the decoding probe (adapter) bound to Recognition Site 1. As shown in FIG. 4B, a second binding staple can comprise a 5′ protruding region that binds to the decoding probe (adapter) bound to Recognition Site 2. The decoding probes can be U-probes comprising toehold regions, as described above. As described above, hybridization of the second decoding probe to Recognition Site 2 (and optionally, hybridization of an additional displacer probe) can displace the first decoding probe and bound nucleic acid origami.

The signal enhancement design, as described above, can combined with in situ sequencing chemistry. Once the RCPs have been generated, the origami structure can be used to either directly hybridize to the RCP, as shown in FIG. 6A, or to an adapter probe which is hybridized to the RCP, as shown in FIG. 6B. Hybridizing the origami structure directly to the RCPs, as shown in FIG. 6A, means that it effectively acts as a decoder probe that is specific to a gene and encodes for a specific fluorescent signal. While this is feasible for a low-plex assay (i.e. 4-8 genes), any use of these structures within sequencing set-ups requiring multiple cycles (6 cycles) would require many origami structures to be produced per cycle (e.g., ˜200 origami structures per cycle). A more straightforward set-up is to have the origami structures encode for a specific fluorescent signal and make the “binding staples” complementary to the binding sites on the adapter probes that typically bind the sequencing probes, as shown in FIG. 6B. Therefore, only 4 Origami structures will need to be created, each encoding for one of the following channels, Cy3, Cy5, AF488, AF750. During the sequencing cycles, the adapter probes are hybridized as normal, then, instead of the sequencing pool, the origami pool (e.g., a mix of origami probes) is added and can then hybridize to the adapter probes on the target nucleic acids, as shown in FIG. 7. This will result in a more intense signal as compared to only hybridizing a single detectably labelled oligonucleotide to the adapter probe. Thus, 4 origamis can be used to decode a large number of different transcripts via sequential hybridization, wherein the specificity comes from the binding regions on the adapter probes and the corresponding binding region of the binding staple. The nucleic acid origamis can be any structure that allows for one or more protruding staples, and need not be geometrically distinct.

In some embodiments, a single nucleic acid origami can be used to decode a nucleotide barcode sequence, wherein the nucleic acid origami comprises four binding staples, and each binding staple uniquely corresponds to a barcode subunit. FIG. 3 shows an exemplary nucleic acid origami structure comprising four binding staples for binding to a target nucleic acid (e.g., a RCP, an intermediate probe or a nucleic acid molecule of the cell). In the origami design shown in FIG. 3, the binding staples are positioned in pairs in Recognition Site 1 or Recognition Site 2. In each of the two sites, one of the protruding staples protrudes from the structure at its 5′ end and the other strand protrudes from its 3′ end. The strand protruding from the 3′ end is able to bind the adapter probes binding to the R1 site on the RCP (FIG. 4A), the strand protruding from the 5′ end is able to bind the adapter probes recognizing the R2 site on the RCP (FIG. 4B). This negates the need to have two separate structures to recognize adapter probes on either site on the RCP, thereby allowing a more time and cost-efficient method. According to this embodiment, the detection staples can comprise a nucleotide barcode sequence corresponding to the target nucleic acid or a barcode of a probe attached thereto. In some embodiments, the detection staples can comprise one or more barcodes (e.g., a pair of barcodes), each barcode comprising two barcode subunits.

C. Detection Design

In some embodiments, the nucleic acid design as shown in FIG. 1B allows detection of a target nucleic acid (e.g., mRNA) without requiring amplification. As shown in FIG. 1B, the nucleic acid origami can be designed with two binding staples that anneal to adjacent regions of the target nucleic acid such that the ends of the two binding staples can optionally be ligated by template-ligation (an “origami-padlock”). The approach taken by the origami-padlock allows for the detection of mRNA directly using only T4 RNA ligase 2 without continuing with the Phi29 amplification step. This further saves in costs for enzymes as well as additional QC that will need to be run and makes the method faster as well as more efficient. Techniques that do not require amplification, such as smFISH or MERFISH, typically require 30-50 different fluorescent probes to target the mRNA. In contrast, as shown in Example 5, the methods provided herein can be performed using 4 or fewer origami-padlocks to detect a target nucleic acid, and do not require amplification in some cases.

In some embodiments, the nucleic acid origami structure can be used to directly detect a nucleic acid molecule, e.g., an mRNA molecule. As shown in FIG. 5, in some embodiments, the nucleic acid origami comprises a binding staple with a protruding 5′ end and a binding staple with a protruding 3′ end. In some embodiments, the 5′ protruding binding staple is designed to have a phosphate group at its 5′ end and the 3′ protruding binding staple has an RNA base at its 3′ end. The two protruding staples hybridize to the RNA in such a fashion as to position the 5′ phosphate group next to the 3′ RNA base of the other staple strand. In some embodiments, four DNA origami structures will be designed per gene, each targeting a specific region of the mRNA molecule.

As discussed above, in some embodiments, ligation of the ends of the two binding staples can stabilize the position of a nucleic acid origami on a target molecule. However, in other embodiments, the nucleic acid origami can directly or indirectly bind to the target nucleic acid via a single binding staple, or via two or more binding staples that are not ligated. In some embodiments, a binding region of a binding staple can hybridize adjacent to an anchor probe, wherein the end of the binding staple and the adjacent end of the anchor probe can be ligated to stabilize hybridization of the binding staple to the target nucleic acid.

The nucleic acid molecule detection design (“origami padlock”) is designed to be independent of existing in situ library preparation methods. The key to this design is the protruding “binding staples”, the 5′ staple with a 5′ phosphate group, and the 3′ staple with an RNA base at its 3′ end. The two staples can hybridize to the nucleic acid molecule directly and function in a similar fashion as the chimeric padlock probe (as shown in FIG. 5). After ligation and subsequent washing steps, the library preparation is complete, and the origami padlock design does not need to be subjected to rolling circle amplification. To amplify the signal generated by the origami padlock, the use of a hybridization complex for signal amplification (e.g., HCR or LO-HCR) is feasible within the design. If a low-plex assay (e.g., 4 genes) will be performed, the protruding detection regions of the detection staples can be designed to act directly as an “initiator strand” for the assembly of the hybridization complex for signal amplification (e.g., the LO-HCR (FIG. 8A) or HCR (FIG. 8B)) chemistry. As shown in FIG. 8A, for the LO-HCR chemistry application, the protruding end of the detection staples will be complementary to the initiator strand (e.g., a linker or a fluorescent oligo). Although the linker strands depicted in FIG. 8A comprise toehold regions, the toehold regions are optional in low-plex embodiments that do not require displacement reactions. As shown in FIG. 8B, for the HCR chemistry application, the initiator strand will be complementary to one of the metastable hairpins. Since the fluorescent channel where the origami padlock will be detected in is hard coded into its design, this set-up restricts the number of genes to be decoded to the number of channels present in the imaging set-up.

If a higher multiplex set-up (e.g., >4-5 genes) is required, the protruding detection region of the detection staples will encode for a gene-specific barcode, as shown in FIG. 9. In some embodiments, the gene-specific barcode can be between about 10 and about 50 nucleotides in length, optionally between about 15 and about 25, e.g., about 20, nucleotides in length. The gene-specific barcode can be hybridized to by a gene-specific adapter probe. This adapter probe comprises a toehold region, an origami binding region capable of hybridizing to the gene-specific barcode of the detection staple, a linker, and a region that will be targetable by a unit of the hybridization complex for signal amplification (e.g., using HCR or LO-HCR chemistry). The toehold region on the adapter probe is present to allow the adapter probe to be removed from the origami padlock without the use of formamide stripping (FIG. 9). In this way, several rounds of sequencing (e.g., 6 cycles) can be performed without the need to remove the nucleic acid origami, as shown in FIG. 9.

Alternatively, the protruding detection region of the detection staples can be used to perform the 2-LSD protocol, as shown in FIG. 10. In this set-up, the protruding detection region comprises two recognition sites that have an overlap region (e.g., two 20 nucleotide recognition sites with a 10 nucleotide overlap region). This would allow a gene specific adapter probe to bind to the first recognition site and then be subjected to the signal enhancement chemistry. After imaging the first cycle, the 2-LSD adapter probe can then be displaced by a second adapter probe which will bind to the second recognition site on the same protruding domain of the detection staple (FIG. 10). Optionally, an additional displacer probe that binds to a toehold region can be used to promote the probe displacement. As with the method described above (FIG. 9), this allows for several rounds of sequencing without the need to remove the origami padlock from the target nucleic acid (e.g., RNA). In addition to this, it allows for a more rapid switching between the sequencing cycles as the existing adapter probe is replaced with the second adapter probe within the same step. In the method shown in FIG. 9, the first adapter probe would have to be replaced first and then the second adapter probe will have to be hybridized in a second step.

D. Decoding Barcode Sequences and 2-LSD

In some aspects of the methods provided herein, the nucleic acid origami is used to detect a nucleotide barcode in a target nucleic acid, e.g., a nucleotide barcode of a RCP (“signal enhancement design”). In other aspects of the methods provided herein a nucleotide barcode sequence can be comprised by or linked to a detection staple, and the binding staple can bind to a region of a target nucleic acid such as an mRNA (“detection design”). Thus, the methods provided herein allow a target nucleic acid to be specifically linked to a barcode sequence and detected. In some embodiments, a nucleotide barcode sequence comprises multiple sequential barcode positions, which can be interrogated separately, and sequentially. The nucleotide barcode sequence is essentially split into multiple sequential barcode positions, each of which comprises at least one barcode subunit. This sequential analysis of multiple barcode positions dramatically increases the coding capacity of the system. Methods of decoding multiple sequential barcode positions have been described, for example, in US2021/0340618, the content of which is herein incorporated by reference in its entirety.

i. Barcodes

When the nucleic acid molecule comprising the nucleotide barcode sequence is contacted with the first set of decoding probes (e.g., adapter probes comprising (i) a target hybridization sequence that hybridizes to a barcode sequence, and (ii) an adapter sequence that hybridizes to a nucleic acid origami), the decoding probe with the sequence complementary to the barcode subunit at the first sequential barcode position can hybridize to the nucleic acid molecule at said first sequential barcode position. After hybridization of the decoding probe, a signal can then be detected from a detectable reporter (e.g., a fluorescently labelled nucleic acid origami) bound to the first decoding probe (e.g., an adapter probe) which has hybridized to the nucleotide barcode sequence at the first sequential barcode position. The adapter sequences of the decoding probes may each correspond to a nucleic acid origami labelled with a different fluorophore, such that each barcode subunit directly corresponds to a single reporter.

The plurality of nucleic acid origami used for sequential decoding of barcode positions can comprise the same nucleic acid scaffold and/or the same staple region(s), but different binding regions. In some embodiments, the binding region of the binding staple corresponds to the fluorescent labels in the detection staples, optionally wherein the plurality of nucleic acid origami comprise 3, 4, 5, or more different fluorescent labels. In this way, a single nucleic acid origami can be used to correspond to a single fluorescent label, and the same nucleic acid origami can be used for multiple different barcode subunits that correspond to that fluorescent label. This design allows a smaller number on nucleic acid origami probes to be used for sequential decoding of a large number of barcode sequences. In some instances, the nucleic acid origami is used to detect a nucleotide barcode in a target nucleic acid, e.g., a nucleotide barcode of a RCP, indirectly via an intermediate adapter probe. In some instances, a temporal order of signals detected from a plurality of nucleic acid origami probes can correspond to an analyte of a plurality of analytes.

A given signal code may be assigned to multiple barcode subunit pairs in the same nucleotide barcode sequence, and/or to multiple barcode subunit pairs in different nucleotide barcode sequences. Each reporter (e.g., nucleic acid origami labelled directly or indirectly with a given fluorescent label) may therefore be observed at multiple barcode positions in a single nucleotide barcode sequence, and/or at a given barcode position in multiple different nucleotide barcode sequences. As indicated above, the identity of the barcode subunits within a given barcode position may be detected from the signal code sequence in order to decode the nucleotide barcode sequence.

ii. Displacer probes

In some embodiments of the methods described herein, it may be desirable to remove hybridized probes in a method for decoding a barcode position without subjecting the sample to harsh probe removal steps such as chemical denaturation (formamide stripping). In some embodiments, it is particularly important to be able to remove certain hybridized probes (e.g., a detectably labelled oligonucleotide, or an intermediate probe or adapter as shown in FIG. 10) without damaging the structure of a nucleic acid origami. Thus, in some embodiments, methods provided herein may involve the use of displacer probes to remove hybridized probes (for example, as shown in FIG. 10).

The hybridization of a subsequent intermediate probe or adapter or detectably labelled probe may constitute a strand displacement reaction, wherein the hybridized preceding detection probe is displaced from the nucleotide barcode sequence. In various examples, the hybridized preceding probe is a nucleic acid origami probe described herein, and the binding region of the nucleic acid origami probe is displaced from its target sequence (e.g., a nucleotide barcode sequence). On other examples, the hybridized preceding probe is a detection probe hybridized to a barcode sequence in a detection region of a detection staple, or in an adapter hybridized to the detection staple. In this strand displacement reaction, the sequence complementary to the subsequent barcode subunit, and the second flanking sequence of the subsequent probe act as a toehold, hybridizing to the nucleotide barcode sequence and allowing the first flanking sequence to invade the adjacent hybrid formed between the preceding probe and the nucleotide barcode sequence at the overlapping region between the preceding barcode position and the barcode position that is being read. This strand invasion by the first flanking sequence of the subsequent probe disrupts the hybridization between the second flanking sequence of the preceding probe and the nucleotide barcode sequence, and thus promotes the displacement of the preceding probe.

The intermediate probe or adapter can comprise a sequence that hybridizes to a target sequence (e.g., a barcode subunit) and an adapter region that hybridizes to the binding region of a nucleic acid origami binding staple. In some embodiments, the intermediate probe or adapter can comprise a sequence that hybridizes to a target sequence in the detection region of a detection staple (as shown in FIG. 10). The detectably labelled probe can be directly or indirectly conjugated to a fluorophore.

In other embodiments, displacer probes can be used to remove a nucleic acid origami probe from a target nucleic acid, e.g. by displacing a binding region of a binding staple that is hybridized to the nucleic acid, or by displacing an adapter that is hybridized to the binding region and to the nucleic acid.

In some aspects, the methods provided herein allow decoding of a barcode sequence comprised by a target nucleic acid using the nucleic acid origami (optionally, together with adapters) as decoding probes, or decoding a barcode sequence comprised by a detection staple using detectably labelled oligonucleotides (optionally, together with adapters), as decoding probes.

In some embodiments, barcode subunits (e.g., subsequences) can be overlapping. In such a method, each barcode position comprises a barcode subunit pair comprising a first barcode subunit and a second barcode subunit, wherein the second barcode subunit from each barcode position at least partially overlaps with the first barcode subunit of the adjacent barcode position in the sequence. The order of barcode subunits (e.g., pairs) in each nucleotide barcode sequence may define a signal code sequence which comprises a signal code corresponding to each barcode subunit pair, and which is distinct from the signal code sequences of other nucleotide barcode sequences and identifies a given nucleotide barcode sequence. In some embodiments, the decoding probes may hybridize not only to a barcode position in the barcode sequence, but also to a general region present in the nucleic acid molecule, outside the barcode sequence. Thus, the nucleic acid molecule may comprise general regions flanking the barcode sequence. The general regions are common to different nucleic acid molecules. In some embodiments, the use of such common regions facilitates a “back-and-forth” decoding method, wherein additional displacer probes are used to assist in the displacement and removal of a preceding decoding probe. The common region can be used to provide a binding site, or a part thereof, for a displacer probe. In particular, rather than using displacer probes which comprise a portion (domain) which is specific for, or complementary to, a barcode position, the displacer probe can be designed to have a domain which is complementary to the common region (as well as a domain complementary to a toehold sequence in a decoding probe). It will be understood that any of the “back-and-forth” detecting or decoding methods disclosed herein may involve the use of displacer probes to assist in the displacement of a preceding decoding probe. Thus, they may be performed using decoding probes in the form of U-probes, which comprise a toehold region for binding of a common displacer probe. Such a method using 2-site decoding probe displacement is termed “2-LSD” herein. Methods using 2-site decoding probe displacement have been described in US20210340618, the content of which is herein incorporated by reference in its entirety.

As outlined above, such U-probes may be designed to hybridize to a given barcode position, or domain within a target nucleotide sequence, or detection region of a nucleic acid origami binding staple, with 2 single-stranded overhangs, one for hybridization of a reporter probe (i.e., the binding staple of a nucleic acid origami in the “signal enhancement” design, a detectably labelled oligonucleotide in the “detection” design) and the other for a displacer probe. Accordingly, a given U-probe may be displaced from both sides simultaneously; on one side by the subsequent U-probe hybridizing to the overlapping region shared between the decoding probe binding sites; and on the other side by the displacer probe hybridizing to the displacer probe overhang. This double displacement reaction is extremely efficient, and thus allows for decoding probes to be switched quickly between sequencing cycles, without the need for chemical stripping (or any of the damage to the sample that is associated therewith). Thus, the nucleic acid origami can remain bound to the sample for multiple rounds of decoding. In some embodiments, the displacer probe overhang (i.e. the displacer toehold overhang) used in the decoding probes may be common for all decoding probes capable of hybridizing to a given binding site. The use of such decoding probes with “back and forth” is particularly advantageous, as such methods involve the use of only 2 decoding probe binding sites, and thus it can be seen that only 2 displacer probe overhangs will be present across all of the decoding probes (one for each binding site). Accordingly, a single displacement probe can be used to simultaneously displace decoding probes bound to an equivalent barcode position from all of the RCPs within a given sample simultaneously (together with the displacement mediated by the subsequent decoding probes). This further increases the efficiency of the method as a whole, and reduces the cost of the method, as fewer different probes are required.

E. Hybridization Complexes and Methods for Signal Amplification

In some embodiments, the method may optionally include use of hybridization complexes and methods for signal amplification (e.g., HCR, linear oligo-HCR, or branched complexes). In some embodiments, the signal amplification is achieved via a non-enzymatic method. In some aspects, any suitable system for assembling hybridization complexes for signal amplification can be used and may include a polymerisation reaction involving a plurality of monomer units. Hybridization chain reaction (HCR) is known in the art as a technique for enzyme-free nucleic acid amplification based on a triggered chain of hybridization of monomer nucleic acid molecules (termed “HCR monomers”) to one another to form a nicked nucleic acid polymer. This polymeric product of the HCR reaction may be generated as a signal which is ultimately detected in order to indicate the presence of a target analyte. In other words, HCR may be used as a signal-generating means to generate a readily detectable signal for detection of a target analyte. In some embodiments, each origami can provide multiple sites for a hybridization complex to form.

HCR was initially described in Dirks and Pierce, 2004, PNAS, 101(43), 15275-15278 and in U.S. Pat. Nos. 7,632,641 and 7,721,721, the contents of each of which are herein incorporated by reference in their entirety, and has subsequently been developed as a detection technique (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401, the contents of each of which are herein incorporated by reference in their entirety). HCR has previously been combined with smFISH and tissue clearing to increase signal to noise ratio (SNR) in mouse brain samples (See, for example, Shah et al 2016, Neuron. 92(2):342-357. doi:10.1016/j.neuron.2016.10.001, which is herein incorporated by reference in its entirety).

HCR is a well-known technique for enzyme-free nucleic acid amplification based on a triggered chain of hybridization of nucleic acid molecules starting from HCR monomers, which hybridise to one another to form a nicked nucleic acid polymer. This polymer is the product of the HCR reaction which is ultimately detected in order to indicate the presence of the target analyte.

In some aspects, HCR monomers comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), i.e. a sequence which is complementary to the exposed toehold region (input domain) of the first HCR monomers. Crucially, however, in the absence of the HCR initiator, these interacting regions are protected by the secondary structure (e.g., they are not exposed), and thus the hairpin monomers are stable or kinetically trapped (also referred to as “metastable”), and remain as monomers (i.e. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g., the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g., all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

It can be seen that the HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerisation reaction would then proceed as described above, except that the resulting product would be a polymer having a repeating unit of first, second and third monomers consecutively. Corresponding systems with larger numbers of sets of HCR monomers could readily be conceived. Branching HCR systems have also been devised and described, and may be used in the methods herein.

The HCR monomers may contain a region of self-complementarity. The self-complementary regions may hybridize to one another to form a region of secondary structure. In some embodiments, the region of secondary structure will contain a loop of single stranded nucleic acid, more particularly a stem-loop or hairpin structure comprising a double stranded “stem” region and a single stranded loop. More particularly, the secondary structure may be a metastable secondary structure. In preferred embodiments, the metastable secondary structure is or comprises a stem-loop, or hairpin.

In the simplest form of HCR, two different types of stable hairpin monomer, referred to here as first and second HCR monomers, undergo a chain reaction of hybridization events to form a long nicked double-stranded DNA molecule when an HCR initiator nucleic acid molecule is introduced. The HCR monomers have or comprise a hairpin structure comprising a double stranded stem region, a loop region connecting the two strands of the stem region, and a single stranded region at one end of the double stranded stem region. The single stranded region which is exposed (and which is thus available for hybridization to another molecule, e.g., initiator or other HCR monomer) when the monomers are in the hairpin structure are known as the “toehold region” (or “input domain”). The first HCR monomers each further comprise a sequence which is complementary to a sequence in the exposed toehold region of the second HCR monomers. This sequence of complementarity in the first HCR monomers is known as the “interacting region” (or “output domain”). Similarly, the second HCR monomers each comprise an interacting region (output domain), e.g., a sequence which is complementary to the exposed toehold region (input domain) of the first HCR monomers.

In the absence of the HCR initiator, these interacting regions are protected by the secondary structure (e.g., they are not exposed), and thus the hairpin monomers are stable or kinetically trapped (also referred to as “metastable”), and remain as monomers (e.g., preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g., the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted, leading to the formation of a nicked chain of alternating units of the first and second monomer species.

In some embodiments, the detection staple may provide a platform for detection of the target nucleic acid via linear oligo-HCR. Linear oligo-HCR (LO-HCR) involves detecting a nucleic acid or non-nucleic acid target analyte by detecting the polymeric product of an HCR reaction which acts as a reporter for the target analyte, wherein the HCR reaction is conducted using HCR monomers which, contrary to conventional hairpin HCR monomers, have a single-stranded linear structure with no hairpin or other metastable secondary structure.

The step of performing a HCR reaction may comprise generating multiple HCR products for each analyte. In particular, multiple HCR products may be generated for the analyte in one HCR reaction that is in one run or cycle of the HCR reaction which is performed. This may be achieved by providing a target nucleic acid molecule comprising multiple (e.g., at least two) copies of a marker sequence, and/or multiple target molecules for each analyte, and/or a HCR initiator capable of initiating multiple HCR reactions (i.e. multiple separate HCR reactions per HCR initiator). In other words, the method may comprise providing at least two marker sequences per target nucleic acid molecule (or in other words, the target nucleic acid molecule may comprise at least 2 copies of a marker sequence), and/or at least two target nucleic acid molecules for a target analyte to be detected, and/or initiating at least two HCR reactions from each HCR initiator. In the case of the latter, the HCR initiator may comprise at least two HCR initiation points (or initiation sites), e.g., at least two initiator domains.

In the HCR reaction, HCR monomers are polymerised to form a HCR product (HCR polymer) by hybridization to one another. In particular, a set of HCR monomers designed to hybridize to one another (for example a set of first and second HCR monomers) are polymerised to form a HCR product. The initiator binds to a first HCR monomer, leading it to bind to second HCR monomer, which in turn binds to another first HCR monomer, and so on in a cascade reaction. This is described further below. HCR monomers designed to hybridize to one another to form a HCR product may be termed as “cognate” HCR monomers or as a HCR monomer set, or HCR monomer system. As noted above, unlike conventional HCR monomers, which comprise a hairpin or other metastable nucleic acid structure, the present method uses HCR monomers which each have a single-stranded linear structure, e.g., which have no secondary structure. In particular, the HCR monomers have no regions of self-complementarity which are capable of forming an intramolecular duplex. In other words, the HCR monomers do not comprise any double-stranded regions, and in particular do not have, contain or comprise any intra-molecular double-stranded region. They do not have any hairpin or stem-loop structure(s). The HCR monomers are single-stranded linear oligonucleotides comprising no regions of duplex, or more particularly no stem-loop structure.

An HCR monomer set may be specific to, or cognate for, a particular HCR initiator sequence, such that the HCR reaction involving that set may be triggered (or initiated) only by a particular HCR initiator. The HCR initiator is provided in one or more parts and may be comprised in the marker sequence in the target nucleic acid molecule, or may hybridize to the marker sequence in the target nucleic acid molecule. Accordingly, the initiation of the HCR reaction is dependent on the presence of the target nucleic acid molecule, and is determined by the marker sequence that is present in the target nucleic acid molecule. In turn, the presence of the target molecule is dependent on the presence and/or amount of the target analyte, or is indicative of the presence and/or amount of the target analyte.

In an embodiment, the HCR monomers for the HCR reaction may be selected or designed so as to generate a HCR product which is distinctive, or indicative, for the analyte. In an embodiment, the HCR product generated for a given analyte may thus be distinguished from a HCR product generated for another analyte. In another embodiment, multiple HCR products may be generated based on the target nucleic acid molecule for a given analyte, and together the multiple HCR products may provide the signal by means of which an analyte is detected, and distinguished. For example, multiple HCR products may be generated in a combinatorial or sequential labelling scheme, as described further below. Thus, for a given analyte, multiple sets of HCR monomers may be provided, each for a separate HCR reaction. (Each set may comprise the monomers necessary for producing a HCR product, e.g. comprising 2 species of HCR monomers cognate for one another, that is which hybridize together to form a HCR product, and different sets may produce distinct, or distinguishable, HCR products).

To perform sequential HCR labelling reactions it may be desirable or in some cases necessary to remove a detected HCR product, before the next cycle is performed (i.e. before the next sequential HCR reaction is initiated). HCR products also need to be removed in methods in which multiple analytes are detected in different cycles. Conventional removal techniques such as formamide stripping may denature or disrupt binding of the nucleic acid origami to the target nucleic acid or adapter. Thus, in some embodiments it is desirable to use less harsh methods and displacement probes may be used, for example invading probes, which invade the hybrid between the target nucleic acid molecule (marker sequence) and the HCR initiator or first HCR monomer, in order to displace the hybridised HCR product. Various such displacement (or displacer) probes have been described, for example the so-called “eraser probes” of Xiao and Guo 2018, Front Cell Dev Biol 6:42, doi 103389/fcell 2018.00042 and Douse et al 2012, NAR 40(7) 3289-3298, which may be adapted for use herein. This may include providing the HCR initiator with a separate displacer-binding toehold domain, which does not hybridize to the target nucleic acid molecule nor to a HCR initiator, and which is available for binding to a displacer probe.

In some embodiments, the first and/or second HCR monomers may comprise an overhang region (i.e. a displacer-binding toehold domain) capable of facilitating a displacement reaction to depolymerise the HCR product. This overhang region may be targeted by displacement probes. Such displacement probes comprise a sequence complementary to the overhang region, and may further comprise a sequence complementary to at least a portion of the input/output domain of the first or second HCR monomer. Accordingly, they can hybridize to the overhang region of the HCR monomers within the HCR product, with the overhang region acting as a toehold, and invade the hybrid between the first and second monomers in the polymeric HCR product, thus leading to the dissociation of the HCR product. This displacement-initiated depolymerisation method may be particularly useful in situations where the method involves the use of an HCR initiator complex capable of supporting multiple HCR reactions. In such situations, the HCR products may be too large to be effectively removed from the target nucleic acid molecule without the use of high temperatures and/or harsh chemical agents, which may damage the sample. Accordingly, breaking up the polymeric chain allows for the HCR product to be more readily removed. In some embodiments, this displacement mechanism may be combined with the use of temperature/chemical agents, as discussed above, in order to facilitate the removal of the HCR product.

In some situations, toehold-mediated displacement may not be necessary in order to displace a preceding HCR product. For example, it may be sufficient to simply rely on equilibrium kinetics, wherein unbound preceding HCR initiators and/or HCR monomers are washed away, and subsequent HCR initiator and/or HCR monomers are added in excess, such that the signal from the subsequent HCR product can be detected at sufficient strength.

F. Signal Detection and Analysis

In some aspects, the methods disclosed herein involve the use of one or detectably labelled nucleic acid origami probes or nucleic acid origami probe sets that directly or indirectly hybridize to a target nucleic acid, such as any of the target nucleic acids described in Section III (See, subsection B. “Signal enhancement design”). In some aspects, the methods disclosed herein involve the use of detection probes or HCR/LO-HCR subunits, wherein the detectably labelled probes or HCR/LO-HCR subunits hybridize directly or indirectly to a detection staple of a nucleic acid origami (See, subsection B “Signal enhancement design”). In some embodiments, methods disclosed herein sample can be contacted, in separate rounds, with different detectably labelled oligonucleotides for analyzing one or more sets in the barcoded probe sets (and thereby analyzing the one or more analyte panels corresponding to the one or more sets). In some embodiments, the detectably labelled oligonucleotides can be changed between rounds while nucleic acid origami remains bound to analytes in the sample.

In some embodiments, a detectably labelled oligonucleotide directly hybridizes to its target, e.g., a transcript or DNA locus. In some embodiments, a detectably labelled oligonucleotide specifically interacts with (recognizes) its target through binding or hybridization to one or more intermediate, e.g., an oligonucleotide, that is bound, hybridized, or otherwise specifically linked to the target. In some embodiments, an intermediate oligonucleotide is a barcoded probe.

In some embodiments, an intermediate oligonucleotide is hybridized against its target with an overhang such that a second oligonucleotide with complementary sequence (“bridge oligonucleotide” or “bridge probe”) can bind to it. In some embodiments, an intermediate oligonucleotide or probe targets a nucleic acid and is optionally labelled with a detectable moiety, and comprises an overhang sequence after hybridization with the target. In some embodiments, an intermediate oligonucleotide or probe comprises a sequence that hybridizes to a target, an overhang sequence, and optionally a detectable moiety. In some embodiments, an intermediate oligonucleotide or probe (e.g., barcoded probes that directly bind analytes in a sample) comprises a sequence that hybridizes to a target and an overhang sequence. In some embodiments, an intermediate oligonucleotide or probe does not have a detectable moiety. In some embodiments, a second oligonucleotide or probe is a detectably labelled oligonucleotide. In some embodiments, a second detectably labelled oligonucleotide is labelled with a dye. In some embodiments, a detectably labelled oligonucleotide is labelled with an HCR polymer. In some embodiments, intermediate oligonucleotides bound to targets are preserved through multiple contacting, removing and/or imaging steps; sequential barcodes are provided through combinations of detectable labels that are linked to intermediate oligonucleotides through bridge probes in the contacting and imaging steps. For example, when detectably labelled oligonucleotides are used as bridge probes, barcodes are provided by detectably labelled oligonucleotides that hybridize with intermediate oligonucleotides through their overhang sequences. After an imaging step, bridge oligonucleotides are optionally removed as described herein. In some embodiments, one intermediate oligonucleotide is employed for a target. In some embodiments, two or more intermediate oligonucleotides are employed for a target. In some embodiments, three or more intermediate oligonucleotides are employed for a target. In some embodiments, four or more intermediate oligonucleotides are employed for a target. In some embodiments, five or more intermediate oligonucleotides are employed for a target.

In some embodiments, each intermediate oligonucleotide hybridizes with a different sequence of a target. In some embodiments, each intermediate oligonucleotide of a target comprises the same overhang sequence. In some embodiments, each detectably labelled oligonucleotide for a target comprises the same sequence complimentary to the same overhang sequence shared by all intermediate oligonucleotides of the target. In some embodiments, an intermediate oligonucleotide comprises a sequence complimentary to a target, and a sequence complimentary to a detectably labelled oligonucleotide.

In some embodiments, each detectably labelled oligonucleotide in a set has a different target, e.g., a transcript or DNA locus. In some embodiments, two or more detectably labelled oligonucleotides in a set have the same target. In some embodiments, two or more detectably labelled oligonucleotides target the same transcript. In some embodiments, two or more detectably labelled oligonucleotides target the same DNA locus. In some embodiments, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90 or 100 detectably labelled oligonucleotides the same target. In some embodiments, two or more detectably labelled oligonucleotides target the same target. In some embodiments, five or more detectably labelled oligonucleotides target the same target.

In some embodiments, all detectably labelled oligonucleotides for a target in a set have the same detectable moieties. In some embodiments, all detectably labelled oligonucleotides are labelled in the same way. In some embodiments, all the detectably labelled oligonucleotides for a target have the same fluorophore. In some embodiments, methods described herein comprise sequentially contacting the sample with multiple sequential sets of detectably labelled oligonucleotides, e.g., for decoding barcode sequences in detection staples of the nucleic acid origami or for decoding a sequence recognized by the binding region of a nucleic acid origami. Thus, detection oligonucleotides for the same target that are used in a separate cycle could be considered to be part of a different set, and could be associated with the same detectable moiety or a different detectable moiety.

In some embodiments, detectably labelled oligonucleotides for a target are positioned within a targeted region of a target. A targeted region can have various lengths. In some embodiments, a targeted region is about 20 bp in length. In some embodiments, a targeted region is about 30 bp in length. In some embodiments, a targeted region is about 40 bp in length. In some embodiments, a targeted region is about 50 bp in length. In some embodiments, a targeted region is about 60 bp in length. In some embodiments, a targeted region is about 80 bp in length. In some embodiments, a targeted region is about 100 bp in length. In some embodiments, a targeted region is about 150 bp in length. In some embodiments, a targeted region is about 200 bp in length. In some embodiments, a targeted region is about 250 bp in length. In some embodiments, a targeted region is about 300 bp in length. In some embodiments, a targeted region is about 350 bp in length. In some embodiments, a targeted region is about 400 bp in length. In some embodiments, a targeted region is about 450 bp in length. In some embodiments, a targeted region is about 500 bp in length. In some embodiments, a targeted region is about 600 bp in length. In some embodiments, a targeted region is about 700 bp in length. In some embodiments, a targeted region is about 800 bp in length. In some embodiments, a targeted region is about 900 bp in length. In some embodiments, a targeted region is about 1,000 bp in length. In some embodiments, detectably labelled oligonucleotides for a target are positioned in proximity to each other on the target.

In some embodiments, targets of one set of detectably labelled oligonucleotides are also targets of another set. In some embodiments, targets of one set of detectably labelled oligonucleotides overlap with those of another set. In some embodiments, the overlap is more than 10%, 20%, 30%, 40%, 50%, 60%, 70%, 80%, 90%, 95%, or 100%. In some embodiments, targets of one set of detectably labelled oligonucleotides are the same as targets of another set. In some embodiments, each set of detectably labelled oligonucleotides targets the same targets.

As used herein, a detectably labelled oligonucleotide is labelled with a detectable moiety. In some embodiments, a detectably labelled oligonucleotide comprises one detectable moiety. In some embodiments, a detectably labelled oligonucleotide comprises two or more detectable moieties. In some embodiments, a detectably labelled oligonucleotide has one detectable moiety. In some embodiments, a detectably labelled oligonucleotide has two or more detectable moiety.

Probes and methods for binding and identifying a target nucleic acid have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety.

In some embodiments, a detectable moiety is or comprises a nanomaterial. In some embodiments, a detectable moiety is or compresses a nanoparticle. In some embodiments, a detectable moiety is or comprises a quantum dot. In some embodiments, a detectable moiety is a quantum dot. In some embodiments, a detectable moiety comprises a quantum dot. In some embodiments, a detectable moiety is or comprises a gold nanoparticle. In some embodiments, a detectable moiety is a gold nanoparticle. In some embodiments, a detectable moiety comprises a gold nanoparticle.

One of skill in the art understands that, in some embodiments, selection of label for a particular probe in a particular cycle may be determined based on a variety of factors, including, for example, size, types of signals generated, manners attached to or incorporated into a probe, properties of the cellular constituents including their locations within the cell, properties of the cells, types of interactions being analyzed, and etc.

For example, in some embodiments, probes are labelled with either Cy3 or Cy5 that has been synthesized to carry an N-hydroxysuccinimidyl ester (NETS-ester) reactive group. Since NETS-esters react readily with aliphatic amine groups, nucleotides can be modified with aminoalkyl groups. This can be done through incorporating aminoalkyl-modified nucleotides during synthesis reactions. In some embodiments, a label is used in every 60 bases to avoid quenching effects.

In some embodiments, sequence analysis of the target and/or barcoded probes can be performed by sequential fluorescence hybridization (e.g., sequencing by hybridization). Sequential fluorescence hybridization can involve sequential hybridization of detection probes comprising an oligonucleotide and a detectable label to a detection region of a detection staple, or an adapter associated with the detection region. In some embodiments, the nucleic acid origami probes themselves can be used as a type of detection probe, wherein the method comprises sequential hybridization of nucleic acid origami probes to a target sequence and/or barcoded probe.

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, each of which is herein incorporated by reference in its entirety.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entirety.

In some embodiments, the barcodes of the detection regions of a nucleic acid origami probe, or adapters hybridized to the detection regions, are targeted by detectably labelled detection oligonucleotides, such as fluorescently labelled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); U.S. Pat. No. 10,457,980 B2; US 2016/0369329 A1; US 2021/0017587 A1; and US 2017/0220733 A1, all of which are herein incorporated by reference in their entirety. In some embodiments, these assays enable signal amplification, combinatorial decoding, and error correction schemes at the same time.

In some embodiments, nucleic acid hybridization can be used for sequencing. These methods utilize labelled nucleic acid decoder probes that are complementary to at least a portion of a barcode sequence. In some embodiments, the decoder probes may comprise a nucleic acid origami or the decoder probes may bind directly or indirectly to a nucleic acid origami. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), each of which is herein incorporated by reference in its entirety.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, each of which is herein incorporated by reference in its entirety.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

### IV. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

(i) Barcode

A “barcode” is a label, or identifier, that conveys or is capable of conveying information (e.g., information about an analyte in a sample). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

(ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

(iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (i.e., can be oligoribonucleotides) and/or deoxyribonucleotide monomers (i.e., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

(v) Splint Oligonucleotide

A “splint oligonucleotide” is an oligonucleotide that, when hybridized to other polynucleotides, acts as a “splint” to position the polynucleotides next to one another so that they can be ligated together. In some embodiments, the splint oligonucleotide is DNA or RNA. The splint oligonucleotide can include a nucleotide sequence that is partially complimentary to nucleotide sequences from two or more different oligonucleotides. In some embodiments, the splint oligonucleotide assists in ligating a “donor” oligonucleotide and an “acceptor” oligonucleotide. In general, an RNA ligase, a DNA ligase, or another other variety of ligase is used to ligate two nucleotide sequences together.

In some embodiments, the splint oligonucleotide is between 10 and 50 oligonucleotides in length, e.g., between 10 and 45, 10 and 40, 10 and 35, 10 and 30, 10 and 25, or 10 and 20 oligonucleotides in length. In some embodiments, the splint oligonucleotide is between 15 and 50, 15 and 45, 15 and 40, 15 and 35, 15 and 30, 15 and 30, or 15 and 25 nucleotides in length.

(vi) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

(x) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe for in situ assay, or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature. For example, detectably labelled features can include a fluorescent, a colorimetric, or a chemiluminescent label attached to an analyte, probe, or bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, C1-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilD18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF® -97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, Mito Tracker® Green, Mito Tracker® Orange, Mito Tracker® Red, NBD (amine), Nile Red, Oregon Green® 488, Oregon Green® 500, Oregon Green® 514, Pacific Blue, PB1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rhol01, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof

(xi) in situ

As used herein, the term “in situ” refers to the detection of a target analyte in its native context, i.e. in the cell or tissue in which it normally occurs. Thus, this may refer to the natural or native localization of a target analyte. In other words, the analyte may be detected where, or as, it occurs in its native environment or situation. Thus, the analyte is not moved from its normal location, i.e. it is not isolated or purified in any way, or transferred to another location or medium etc. Typically, this term refers to the analyte as it occurs within a cell or within a cell or tissue sample, e.g. its native localization within the cell or tissue and/or within its normal or native cellular environment. In particular, in situ detection includes detecting the target analyte within a tissue sample, and particularly a tissue section. In other embodiments the method can be carried out on a sample of isolated cells, such that the cells are themselves are not in situ.

### EXAMPLES

The following examples are included for illustrative purposes only and are not intended to limit the scope of the present disclosure.

### Example 1: Designing a Spherical DNA Origami Structure

This example demonstrates a method for the design and construction of spherical polyhedral mesh DNA origami structure products. Exemplary DNA origami structures are shown in FIGS. 1A & 1B.

The main spherical DNA core structure has been previously described in Benson et al., Nature 523, 441-444 (2015), the content of which is incorporated herein by reference in its entirety. In some examples described for use in the methods herein, the core spherical wireframe structure has been extensively modified by adding two Recognition sites, each comprising two protruding oligonucleotide strands (e.g., protruding staples). One of the protruding staples protrudes from the structure at its 5′ end, while the other protruding staple protrudes from its 3′ end. For the signal enhancement design, both protruding staples are 22 nucleotides (nt) in length. Specifically, 20 nt of this sequence are complementary to either the rolling circle amplification product (RCP) or an adapter probe, and is connected to the remainder of the protruding staple strand by a 2 nt linker. For the origami padlock design, the two binding staples which protrude from the structure from their 5′ end have a 5′ phosphate group and the two binding staples which protrude from the structure from their 3′ end comprise an RNA base at their 3′ end. The protruding detection staples that provide a complementary sequence to specific detectably labelled oligonucleotides or probes or adapter probes comprises a 22-32 nt protruding 5′ end, which includes a 20-30 nt binding domain (e.g., for the detectably labelled oligonucleotide) with a 2 nt linker.

In one example, the spherical wireframe structure, as described, has been redesigned to include 41 modified DNA strands (“staples”) that protrude from the main structure and can be used to hybridize to their binding sites on the RCP or on the nucleic acid molecule or can be used as target sites for either detectably labelled fluorescent oligonucleotides or HCR or LO-HCR components.

The 41 staples on the wireframe structure include 37 modified staples (“detection staples”) that are a target for detectably labelled oligonucleotides or HCR/LO-HCR chemistry. These 37 detection staples protrude from the main structure at their 5′ end. The protruding ends of the staples are 22 nt in length, 2 nt linker and an additional 20 nt DNA sequence. For the signal enhancement design, this 20 nt sequence is complementary to specific detectably labelled oligonucleotides. For the RNA targeting design, the 20 nt sequence can either be complementary to a specific oligonucleotide that will act as the initiator for the LO-HCR chemistry, or be designed to act as an initiator for the HCR hairpins (FIG. 2).

The additional four protruding staples that will bind to either the RCP, an intermediate probe or a nucleic acid molecule (“binding staples”), are positioned in pairs in Recognition site 1 (R1) or Recognition site 2 (R2), as shown in FIG. 3. In each of the two sites, one of the protruding staples protrudes from the structure at its 5′ end and the other strand protrudes from its 3′ end. For use with the signal enhancement design, the protruding end of the binding staple has a 2 nt linker followed by a 20 nt sequence either complementary to the RCP or an intermediate adapter probe. The different polarities of the binding staples within this design allow the structure to be compatible with 2-LSD design which utilizes displacement of probes. The strand protruding from the 3′ end is able to bind the adapter probes binding to the R1 site on the RCP, the strand protruding from the 5′ end is able to bind the adapter probes recognizing the R2 site on the RCP. This negates the need to have two separate structures to recognize adapter probes on either site on the RCP (FIG. 4).

To utilize the DNA origami structure to directly detect a nucleic acid molecule, the protruding ends of the binding staples are 22 nt long, a 2 nt and a 20 nt sequence that would bind the nucleic acid molecule. The 5′ protruding staple is designed to have a phosphate group at its 5′ end and the 3′ protruding staple has an RNA base at its 3′ end. The two protruding staples hybridize to the RNA in such a fashion as to position the 5′ phosphate group next to the 3′ RNA base of the other staple strand (FIG. 5). This mechanism thus functions in a similar way as a chimeric padlock probe which hybridizes to a target sequence and is ligated and circularized. In some cases, four DNA origami structures will be designed per gene, each targeting a specific region of the mRNA molecule.

In addition to the protruding staple strands, the structure comprises a further 91 “core staples”, which are common to all structures and provide the main structure of the DNA origami structure. The staple strands (e.g., the protruding staples and core staples) hybridize to the scaffold DNA strand to form the final spherical structure.

### Example 2: Folding and Purification of a DNA Origami Structure

This example demonstrates the folding and purification of a DNA origami structure based on a p7249 scaffold DNA (IDT).

The staple strands, including the binding staples, detection staples, and core structure staples, were resuspended in TE buffer at 100 μM concentration and were pooled together to a final stock concentration of 463 nM each. The folding reaction consisted of 20 nM of p7429 DNA scaffold and 200 nM of staple strand mix diluted in 1× PBS, pH 7.4. The folding reaction was placed in a PCR machine and subjected to rapid heat denaturation at 80° C. for 5 min followed by slow cooling from 80° C. to 60° C., over 20 min (1° C./min). The reaction was then allowed to cool from 60° C. to 24° C., over 14 h (1° C./24 min).

The folding reaction was then purified. Excess staples were removed by washing the DNA origami structures repeatedly with PBS, pH 7.4 in 100 kDa MWCO 0.5-ml Amicon centrifugal filters. Filters were passivated with 450 μl 5% Pluronic-F127 in 1× PBS, pH 7.4 overnight at 4° C. and rinsed 5 times with 450 μl DEPC-MQ water, followed by five quick rinses with 450 μl PBS, pH 7.4 before use. In order to purify the origami structures, the folding reaction was added to the rinsed Amicon centrifugal filter and brought to 450 μl final volume with PBS, pH 7.4. The column was centrifuged at 14,000×g at 15° C. for 2 min. The flow-through was discarded and the sample in the column was diluted to 450 μl with PBS, pH 7.4 and centrifuged again. This process was repeated 5-6 times. After the dilution and centrifugation cycles, the column was inverted into a clean collection tube and centrifuged at 1000×g for 2 min. The concentration of the structures was determined via Nanodrop/Qubit and adjusted accordingly.

To assess the proper folding of the DNA origami structures, gel electrophoresis was used. The agarose gel was cast using 2% agarose in 0.5× Tris/borate/EDTA and 10 mM MgCl₂. The structures were loaded onto the gel and run on ice for 2-4 h at 70 V.

### Example 3: Signal Enhancement of Rolling Circle Amplification Products

This example demonstrates a method for the signal enhancement of rolling circle amplification products (RCPs) using a spherical DNA origami structure.

Once the RCPs have been generated, the origami structure can be used to either directly hybridize to the RCP or to an adapter probe which is hybridized to the RCP (FIGS. 6A and 6B). Hybridizing the origami structure directly to the RCPs means that it effectively acts as an adapter probe that is specific to a gene and encodes for a specific fluorescent signal. While this is feasible for a low-plex assay (e.g., 4-8 genes), any use of these structures within the current sequencing set-up (6 cycles) will be unfeasible as 200 origami structures will have to be produced per cycle. In some cases, the origami structures encode for a specific fluorescent signal and make the “binding staples” complementary to the binding sites on the adapter probes that typically bind the detectably labelled oligonucleotides. Therefore, only 4 Origami structures will need to be created, each encoding for one of the following channels, Cy3, Cy5, AF488, AF750. During the sequencing cycles, the adapter probes are hybridized as normal, then, instead of the sequencing pool, the origami pool is added and can then hybridize to the adapter probes on the RCPs (FIG. 7). This will result in a more intense signal as compared to only hybridizing a single detectably labelled oligonucleotide to the adapter probe.

Mouse Tissue Section Preparation

Mice (C57BL/6 strain) at 30 days age (P30) were euthanized and the olfactory bulb was dissected via cryosectioning. Cryosectioning was performed on ThermoFisher cryostat, at 10 μm thickness. Sections were then adhered onto ThermoFisher Superfrost glass slides and stored at −70° C. until processing.

RCA Generation In Situ

The tissue slide was removed from −70° C. storage and allowed to thaw for 5 min at room temperature (RT). Fixation was then performed by incubating the slides in 3.7% PFA in 1× DEPC-PBS at RT for 5 min. The slide was then washed in 1× DEPC-PBS for 1 min at RT. This ensures that the PFA is completely removed before moving to the permeabilization step. The tissue sections were then permeabilized using 0.1M HCl in DEPC-H₂O for 1 min at RT and subsequently quickly washed twice in 1× DEPC-PBS. Following this, the slides were dehydrated with an ethanol series in 70% and 100% ethanol for 2 min, respectively, before the slides were air-dried for 5 min at RT. A Secure Seal Chamber (Grace Bio Labs) was applied to each section, and the sections were rehydrated with a wash buffer before continuing with the reverse transcription step.

A 45 μL of a reaction mix and 5 μL of chimeric probes (100 nM) were added to the secure seal chambers and incubated overnight at 37° C. The probe hybridization mix was removed, and the chambers were washed twice with DEPC-PBS-T. A wash buffer (46 μL) was added to the secure seal chambers and incubated at 37° C. for 30 min. The wash buffer was subsequently removed from the secure seal chambers, and the chambers were washed 3 times with DEPC-PBS-T before continuing with the ligation step.

A reaction mix and enzymes for ligation were added to the secure seal chambers and incubated at 37° C. for 2 h. The mixture as removed from the chamber, and the chamber was washed twice with DEPC-PBS-T before continuing with the amplification step.

A 43.0 μl of reaction mixture and 5 μl of the enzyme (Φ29 Polymerase) were added to the secure seal chambers and incubated at either 37° C. for 3 h or at 30° C. overnight. The amplification reaction mix was removed, and chambers were washed twice with DEPC-PBS-T. Next, the secure seal chambers were removed and the slides were dehydrated with an ethanol series in 70% and 100% ethanol for 2 min respectively before the slides were air-dried for 5 min at RT. The sections were then used for in situ sequencing using the LSD design.

In Situ Sequencing of RCPs in Tissue Sections using DNA Origami as Signal Enhancement

The sections were rehydrated with 2× SSC and the first probe mix was added at 100 nM in basic hybridization buffer (5× SSC+30% Formamide), and incubated at 1 h at 20-37° C. The sections were washed twice with basic washing buffer (1× PBS in DEPC-H₂O). The DNA origami structure was then incubated with a 5-fold molar excess of fluorescent oligonucleotides for 1 h at 37° C.

The DNA origami structures comprising fluorescent oligonucleotides were hybridized in a standard hybridization buffer (5× SSC, 30% Formamide) for 30-60 min at 37° C. After incubation, the hybridized DNA origami structures were washed twice with PBS, pH 7.4. An etOH series (2 min in 70% etOH and 2 min in 100% etOH) was performed, and the structures were mounted with SlowFade, and imaged.

### Example 4: Nucleic Acid Detection

This example demonstrates a method for nucleic acid detection (e.g., RNA, DNA, cDNA, or RCPs) using a spherical DNA origami structure (e.g., origami padlock).

The nucleic acid molecule detection design (“origami padlock”) is designed to be independent of existing in situ library preparation methods. Using this design, the protruding “binding staples”, the 5′ staple with a 5′ phosphate group, and the 3′ staple with an RNA base at its 3′ end. The two staples can hybridize to the nucleic acid molecule directly and function in a similar fashion as a chimeric padlock probe (FIG. 5), where the ends are ligated using the same reaction mixture. After ligation and subsequent washing steps, the library preparation is complete, and the origami padlock design does not need to be subjected to rolling circle amplification. To amplify the signal generated by the origami padlock, the use of either HCR or LO-HCR is feasible within the design. If a low-plex assay (e.g., 4 genes) will be performed, the protruding ends of the detection staples can be designed to act directly as an “initiator strand” for either the HCR or the LO-HCR chemistry (FIG. 8A-B). For the HCR chemistry, the initiator strand will be complementary to one of the metastable hairpins. For the LO-HCR chemistry application, the protruding end of the detection staples will be complementary to the initiator strand (e.g., a linker or a fluorescent oligo). Since the fluorescent channel where the origami padlock will be detected in is hard coded into its design, this set-up restricts the number of genes to be decoded to the number of channels present in the imaging set-up.

If a higher multiplex set-up (e.g., >4-5 genes) is required, the protruding part of the detection staples will encode for a gene-specific barcode. This barcode will be 20 nt in length and can be hybridized to by a gene-specific adapter probe. This adapter probe consists of a 10 nt toehold region, a 20 nt origami binding region, a 2 nt linker and a 20-30 nt region that will be targetable by either the HCR or LO-HCR chemistry. The toehold region on the adapter probe is present to allow the adapter probe to be removed from the origami padlock without the use of formamide stripping (FIG. 9). In this way, several rounds of sequencing (e.g., 6 cycles) can be performed without the need to remove the origami padlock.

Alternatively, the protruding domain of the detection staples can be extended to 30 nt to perform the 2-LSD protocol. In this set-up, the protruding domain would consist of two 20 nt recognition sites that have a 10 nt overlap. This would allow a gene specific adapter probe to bind to the first recognition site and then be subjected to the signal enhancement chemistry. After imaging the first cycle, the 2-LSD adapter probe can then be displaced by a second adapter probe which will bind to the second recognition site on the same protruding domain of the detection staple (FIG. 10). As with the method listed above, this allows for several round of sequencing without the need to remove the origami padlock from the RNA. In addition to this, it allows for a more rapid switching between the sequencing cycles as the existing adapter probe is replaced with the second adapter probe within the same step. In the above method, the first adapter probe would have to be replaced first and then the second adapter probe will have to be hybridized in a second step.

Mouse Tissue Section Preparation

Mice (C57BL/6 strain) at 30 days age (P30) were euthanized and the olfactory bulb was dissected via cryosectioning. Cryosectioning was performed on ThermoFisher cryostat, at 10 μm thickness. Sections were then adhered onto ThermoFisher Superfrost glass slides and stored at −70° C. until processing.

Fixation and Permeabilization

The tissue slide was removed from −70° C. storage and allowed to thaw for 5 min at room temperature (RT). Fixation was then performed by incubating the slides in 3.7% PFA in 1× DEPC-PBS at RT for 5 min. The slide was washed in 1× DEPC-PBS for 1 min at RT. This ensures that the PFA is completely removed before moving to the permeabilization step. The tissue sections were then permeabilized using 0.1M HCl in DEPC-H₂O for 1 min at RT and subsequently quickly washed twice in 1× DEPC-PBS. Following this, the slides were dehydrated with an ethanol series in 70% and 100% ethanol for 2 min, respectively, before the slides were air-dried for 5 min at RT. A Secure Seal Chamber (Grace Bio Labs) was applied to each section, and the sections were rehydrated with 1× DEPC-PBS-T before continuing with the reverse transcription step.

DNA Origami Hybridization

A reaction mix and origami padlocks probes (5-10 nM) were added to the secure seal chambers and incubated overnight at 37° C. The probe hybridization mix was removed, and the chambers were washed twice with DEPC-PBS-T. A wash buffer (46 μL) was added to the secure seal chambers and incubated at 37° C. for 30 min. The wash buffer was subsequently removed from the secure seal chambers, and the chambers were washed 3 times with DEPC-PBS-T before continuing with the ligation step.

Probe Ligation

A reaction mix and enzymes for ligation were added to the secure seal chambers and incubated at 37° C. for 2 h. The mixture was removed from the chamber, and the chamber was then washed twice with DEPC-PBS-T. The secure seal chambers were removed, and the section was dehydrated with an ethanol series in 70% and 100% ethanol for 2 min, respectively, before the slides are air-dried for 5 min at RT. The sections were then be prepared for in situ sequencing.

Adapter Probe Hybridization

The sections were rehydrated with 2× SSC and the adapter probe mix was added at 100 nM in basic hybridization buffer (5× SSC+30% Formamide), and incubated for 1 h at 20-37° C. The sections were washed twice with basic washing buffer (1× PBS in DEPC-H₂O). The origami padlock was then decoded (e.g., read out) using either hybridization chain reaction (HCR) or LO-HCR chemistry. Standard detection oligonucleotides (DOs) may be allowed to hybridize to the origami padlock. In order to detect the signal from these DOs, a 100× oil objective was used, with an exposure time of 1-3 sec.

### Example 5: DNA Origami Structures are Effective for the Detection of PCP4 mRNA

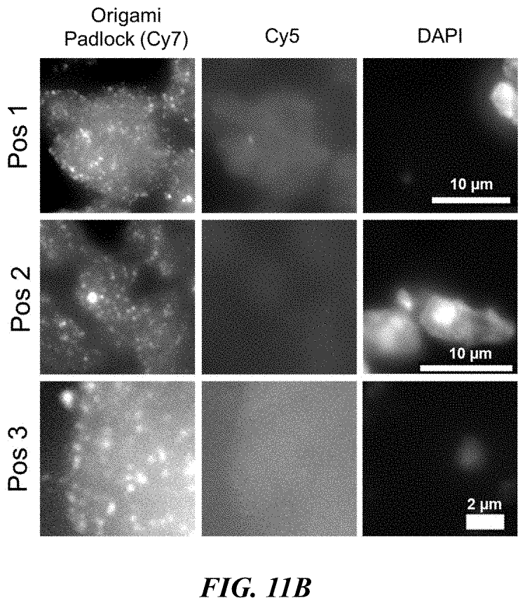

This example demonstrates the detection of a PCP4 mRNA using a spherical DNA origami structure. Four origami padlocks were designed and constructed as described, each corresponding to a unique recognition site of the PCP4 mRNA molecule. The origami padlocks were folded, purified, and run on a 1.5% agarose gel as described. Three of the origami structures were observed to fold properly (FIG. 11A). The structures were pooled together and used at a final concentration of 2.5 nM. The origami padlocks were then added and hybridized to the tissue section as outlined. A Cy7 DO was hybridized to the structures, with a theoretical limit of 37 DOs bound to each origami-padlock.

The tissue was then imaged using a 100× NA 1.3 to visualize the fluorescence signal from the origami padlocks. From the images provided in FIG. 11B, the origami padlocks were able to be detected above background level and appeared as circular spots. This signal is not present in the Cy5 channel, indicating that this is true signal and not autofluorescence signal originating from the tissue section.

As outlined in the description above, the technique using origami probes is designed as a novel method of signal enhancement as well as an RNA detection method. The principle that the Origami design contains protruding strands that can target either an RCP sequence or can be redesigned to act as a chimeric “padlock” all while containing the bulk of the DNA origami structure remains the same between the two designs shows the flexibility of the design, i.e. the bulk of the staple strands and scaffold is identical regardless of the gene that is being targeted or the position on the mRNA that the origami-padlock binds to. The additional 37 protruding strands act as binding surfaces for fluorescently tagged oligonucleotides, LO-HCR chemistry or HCR chemistry. As shown in FIG. 11B, the 37 fluorescently labelled oligos attached to the origami-padlock can be visualized when only 4 origami-padlocks are present on the mRNA molecule. This is not the case with most techniques that target RNA directly. Techniques that do not require amplification, such as smFISH or MERFISH, require 30-50 fluorescent probes to target the mRNA. In techniques where amplification is needed, 3-5 probes are needed. This method only requires 4 origami-padlocks and can be detected without a need for amplification. However, the origami-padlocks can also be designed to be compatible with existing methods such as LO-HCR or HCR which would boost signal intensity and make it more easily detectable.

The approach taken by the origami-padlock allows for the detection of mRNA directly using only T4 RNA ligase 2 without continuing with the Phi29 amplification step. This further saves in costs for enzymes as well as additional QC that will need to be run and makes the method faster as well as more efficient.

The signal enhancement structures allow for the enhancement of the signal of RCPs by a theoretical 37-fold as it would occupy the binding site of the original fluorescent oligo on the RCP and effectively replaces it with 37 fluorescent oligos. In this way, the structure can be used as a signal amplifier. This can be useful as it potentially means that RCPs can be detected on a 10× or even 5× objective which would speed up imaging time dramatically.

In conclusion, the use of DNA origami in the detection of RNA directly or for enhancing the signal of RCPs allows for the RNA targeting portion and the signal enhancement portion to exist on the same structure. In addition, the structure can also be made compatible with amplifying techniques, such as LO-HCR or HCR.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1-101. (canceled)

102. A method for analyzing a biological sample, comprising:
a) contacting a biological sample comprising a plurality of cells with a nucleic acid scaffold and a binding staple,
wherein a nucleic acid origami comprising the nucleic acid scaffold and the binding staple is formed, and
wherein the binding staple comprises a binding region that directly or indirectly binds to a nucleic acid molecule in the biological sample; and
b) detecting the nucleic acid origami in the biological sample, thereby analyzing localization of the nucleic acid molecule in the biological sample.

103. The method of claim 102, wherein the nucleic acid origami comprises a folded core comprising the nucleic acid scaffold, and the binding region protrudes from the folded core.

104. The method of claim 102, wherein the nucleic acid origami is contacted with a detection staple directly or indirectly labelled with a detectable moiety.

105. The method of claim 104, wherein the nucleic acid scaffold forms a folded core of the nucleic acid origami and the detection staple comprises a detection region protruding from the folded core.

106. The method of claim 102, wherein the binding region indirectly binds to the nucleic acid molecule in the biological sample.

107. The method of claim 106, wherein the binding region directly hybridizes to an adapter which directly or indirectly binds to the nucleic acid molecule in the biological sample.

108. The method of claim 102, wherein the nucleic acid molecule is an endogenous DNA or RNA molecule in the biological sample.

109. The method of claim 102, wherein the nucleic acid molecule in the biological sample is comprised in a labelling agent that directly or indirectly binds to an analyte in the biological sample, or is comprised in a product of the labelling agent.

110. The method of claim 109, wherein the nucleic acid molecule in the biological sample is a rolling circle amplification (RCA) product generated in situ using a circular or circularizable probe or probe set that hybridizes to a DNA or RNA molecule in the biological sample.

111. A method for analyzing a biological sample, comprising:
a) contacting a biological sample comprising a plurality of cells with a nucleic acid origami, wherein:
the nucleic acid origami comprises a nucleic acid scaffold, a binding staple, and a plurality of fluorescently labelled detection staples, and
the binding staple comprises a binding region that directly or indirectly binds to a nucleic acid molecule in the biological sample; and
b) detecting fluorescent signals from the plurality of fluorescently labelled detection staples of the nucleic acid origami in the biological sample,
thereby analyzing localization of the nucleic acid molecule in the biological sample.

112. The method of claim 111, wherein one or more of the detection staples are covalently coupled to a fluorophore; and/or comprise a detection region protruding from a folded core comprising the nucleic acid scaffold of the nucleic acid origami.

113. The method of claim 111, wherein the nucleic acid molecule is a rolling circle amplification (RCA) product.

114. The method of claim 113, wherein the RCA product comprises a barcode sequence corresponding to an analyte in the biological sample.

115. The method of claim 114, further comprising analyzing the barcode sequence using sequential hybridization, sequencing by hybridization, sequencing by ligation, sequencing by synthesis, sequencing by binding, or any combination thereof.

116. A method for analyzing a biological sample, comprising:
a) contacting a biological sample comprising a plurality of cells with a plurality of nucleic acid origami, wherein:
the biological sample comprises a plurality of nucleic acid molecules,
each nucleic acid origami comprises a nucleic acid scaffold, a binding staple, and a plurality of fluorescently labelled detection staples,
wherein the binding staple comprises (i) a staple region that hybridizes to the nucleic acid scaffold, (ii) a binding region that hybridizes to an adapter which in turn hybridizes to a target sequence in the plurality of nucleic acid molecules; and
b) detecting fluorescent signals from the fluorescently labelled detection staples,
thereby analyzing localization of the plurality of nucleic acid molecules in the biological sample.

117. The method of claim 102, further comprising repeating the contacting and detecting sequentially one or more times with a different plurality of nucleic acid origami.

118. The method of claim 117, wherein the method comprises sequential detecting of two or more nucleic acid origami at a position in the repeated detecting steps, and the detected signals are used to build a signal code sequence corresponding to localization of the nucleic acid molecule at the position in the biological sample.

119. The method of claim 116, wherein two or more of the plurality of nucleic acid origami and/or of the two or more nucleic acid origami comprise different binding regions.

120. The method of claim 104, wherein the detecting step comprises:
i) contacting the biological sample with a plurality of hybridization chain reaction (HCR) or linear oligo hybridization chain reaction (LO-HCR) monomers, wherein:
one or more HCR or LO-HCR monomers are detectably labelled,
the detection region comprises or is directly or indirectly coupled to an initiator sequence that hybridizes to an HCR or LO-HCR monomer of the plurality to initiate an HCR or LO-HCR, and
an HCR or LO-HCR complex comprising the one or more detectably labelled HCR or LO-HCR monomers is generated; and
ii) detecting a signal from the HCR or LO-HCR complex in the biological sample.

121. The method of claim 120, wherein the detection region hybridizes to an adapter which: (i) hybridizes to an initiator comprising the initiator sequence; or (ii) comprises the initiator sequence.
